(19) United States
(12) Patent Application Publication
(10) Pub. No.: US 2017/0198292 A1
(43) Pub. Date: Jul. 13, 2017

(54) Methods and Compositions for Modulating Lung Cancer Tumor Initiating Cells (TIC), and Oxytocin Receptor (OXTR) Modulatory Agents for Use in Practicing the Same

(71) Applicant: The Board of Trustees of the Leland Stanford Junior University, Stanford, CA (US)

(72) Inventors: Hua Fan-Minogue, Milpitas, CA (US); Atul J. Butte, Menlo Park, CA (US); Jason Wheeler, Tucsom, AZ (US)

(21) Appl. No.: 15/324,164

(22) PCT Filed: Jul. 6, 2015
(86) PCT No.: PCT/US2015/039220

§ 371 Date: Jan. 5, 2017

Related U.S. Application Data
(60) Provisional application No. 62/021,568, filed on Jul. 7, 2014.

(51) Int. Cl. C12N 15/113; C12Q 1/68; G01N 33/574; A61K 45/06

(52) U.S. Cl. CPC C12N 15/1136; A61K 45/06; C12Q 1/6886; G01N 33/57423; C12N 2310/14; C12N 2320/31; C12Q 2600/158; C12Q 2600/106; G01N 2800/52

(57) ABSTRACT

Methods of modulating lung cancer, e.g., squamous cell carcinoma (SQCC), tumor initiating cells (TIC) are provided. Aspects of the methods including contacting a TIC with an OXTR modulatory agent, e.g., an inhibitory agent, in a manner sufficient to modulate the TIC. Aspects of the invention further include compositions that find use in practicing methods of the method. The methods and compositions find use in a variety of different applications, including but not limited to the treatment of SQCC.

### CROSS-REFERENCE TO RELATED APPLICATIONS

Pursuant to 35 U.S.C. §119(e), this application claims priority to the filing date of the U.S. Provisional Patent Application Ser. No. 62/021,568, filed Jul. 7, 2014; the disclosure of which is herein incorporated by reference.

### GOVERNMENT RIGHTS

This invention was made with Government support under contract CA138256 awarded by the National Institutes of Health. The Government has certain rights in the invention.

### INTRODUCTION

Lung cancer is an intractable disease, with a five-year survival at all stages under 16% (Rivera et al., “Lung cancer stem cell: new insights on experimental models and preclinical data,” Journal of Oncology (2011), 549181; Goldstraw et al., “Non-small-cell lung cancer,” Lancet (2011) 378: 1727-1740). Relapses are frequent, as are metastases to nearby tissue or other parts of the body (Lu et al., 78: Cancer of the Lung. Holland-Frei Cancer Medicine (8th ed.) People's Medical Publishing House (2010)). The two main types of lung cancer, small cell lung carcinoma (SCLC) and non-SCLC (NSCLC), are histologically and molecularly distinctive. SCLC, accounting for 15% of lung cancers, has a subtype of small cell carcinoma, which derives from neuroendocrine cells with expression of neuroendocrine (NE) markers and production of ectopic hormones (Giangreco et al., “Lung cancer and lung stem cells: strange bedfellows?” American journal of respiratory and critical care medicine (2007) 175: 547-553). NSCLC, accounting for 85% of lung cancers, has three subcategories: adenocarcinoma (ADC), squamous cell carcinoma (SQCC), and large cell carcinoma (Collins et al., “Lung cancer: diagnosis and management. American family physician 75, 56-63 (2007)). ADC and SQCC account for ˜40% and ˜20% of lung cancers, respectively. They are not considered to be of NE origin (Friedmann et al., “Vasopressin and oxytocin production by non-neuroendocrine lung carcinomas: an apparent low incidence of gene expression,” Cancer letters (1993) 75:79-85), although more recent pathological classification has recognized some subtype of NSCLC with NE morphology and positive NE markers (Travis et al., “New pathologic classification of lung cancer: relevance for clinical practice and clinical trials,” Journal of clinical oncology: official journal of the American Society of Clinical Oncology (2013) 31: 992-1001).

Not surprisingly, a main challenge facing therapy development for lung cancer is its extreme histologic and genetic heterogeneity. Recent advances in high throughput molecular profiling have characterized genomic features of lung cancers. For example, differential recurrent mutations and altered pathways have been identified between the subtypes (Sekido et al., “Molecular genetics of lung cancer,” Annual review of medicine (2003)54:73-87); Minna et al., “Focus on lung cancer,” Cancer cell (2002) 1:49-52; “The-Cancer-Genome-Atlas-Research-Network. Comprehensive genomic characterization of squamous cell lung cancers,” Nature 2012) 489:519-525). EGFR and KRAS mutations primarily occur in ADC, while FGFR1 amplification and DDR2 mutations are mainly found in SQCC (Weiss et al., “Frequent and focal FGFR1 amplification associates with therapeutically tractable FGFR1 dependency in squamous cell lung cancer,” Science translational medicine (2010) 2: 62ra93; Hammerman et al., “Mutations in the DDR2 kinase gene identify a novel therapeutic target in squamous cell lung cancer,” Cancer discovery (2011) 1: 78-89). Inhibitors of these targets can reduce tumor burden, yet their effects on tumor recurrence and metastasis are unclear.

Increasing amount of studies have identified a sub-fraction of tumor cells that appear to self-renew and give rise to differentiated tumor cells, so called tumor initiating cells (TICs) (Eramo et al., “Lung cancer stem cells: tools and targets to fight lung cancer,” Oncogene (2010) 29: 4625-4635). Some researchers believe TICs may be responsible for tumorigenesis, metastasis and drug resistance in solid cancers (Clarke et al., “Cancer stem cells—perspectives on current status and future directions: AACR Workshop on cancer stem cells,” Cancer research (2006) 66:9339-9344; Wang et al., “Cancer stem cells: lessons from leukemia,” Trends in cell biology (2005) 15: 494-501), including lung cancers (Eramo et al., “Identification and expansion of the tumorigenic lung cancer stem cell population,” Cell death and differentiation (2008) 15: 504-514). These ideas make it attractive to target lung TICs as the key to improving prognoses in lung cancer (Giangreco, supra; Eramo (2010), supra). Various markers, such as CD133 (Eramo (2008) supra), CD44 (Leung et al., “Non-small cell lung cancer cells expressing CD44 are enriched for stem cell-like properties,” PloS one (2010) 5, e14062), and CD166 (Zhang et al, “Glycine decarboxylase activity drives non-small cell lung cancer tumor-initiating cells and tumorigenesis,” Cell (2012) 148: 259-272), are used to sort lung TICs populations in different studies. Most of these markers, however, are also expressed by TICs of other cancer types, as well as normal cells (Eramo (2010) supra). Their role in TIC associated tumorigenesis is either unclear or inert (Zhang, supra). These facts underline the necessity of finding markers specific for lung TICs, although current high throughput molecular profiling is based of heterogeneous cell populations, where the TIC signal is extremely diluted (Eramo (2010) supra).

### SUMMARY

Methods of modulating lung cancer, e.g., squamous cell carcinoma (SQCC), tumor initiating cells (TIC) are provided. Aspects of the methods including contacting a TIC with an OXTR modulatory agent, e.g., an inhibitory agent, in a manner sufficient to modulate the TIC. Aspects of the invention further include compositions that find use in practicing methods of the method. The methods and compositions find use in a variety of different applications, including but not limited to the treatment of SQCC.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following FIG.s.

FIG. 1. Most frequently used putative surface markers of lung TICs. (a) Histogram showing the CD-prefixed surface markers occurred in PubMed literatures that have lung TIC as a major topic. CD133, CD44, CD24 and CD34 are the four top markers occurred in both titles and abstracts. (b) Occurrence frequency of these markers from 199 lung TIC related PubMed literatures.

FIG. 2. A function-specific association study identifies robust gene expression correlation between CD133 and OXTR. (a) Schematic overview of the Study. TCGA: The Cancer Genome Atlas. TIC: tumor initiating cells. FACS: fluorescence-assisted cell sorting. (b) Heatmap of gene expression correlation with most used lung TIC markers, PROM1 (CD133), CD44 and CD34, in squamous carcinoma (SQCC), adenocarcinoma (ADC) and normal tissue of lung (q≦0.1, |r|≧0.3). Top 40 genes associated with PROM1 (right) include several receptor genes (bold), including OXTR (bold red). (c) Meta-analysis of the gene expression correlation between OXTR and CD133 using GEO datasets of SQCC.

FIG. 3. Co-expression of CD133 and OXTR in SQCC tumor tissues, cell lines and primary cells. (a) Co-immunofluorescence (IF) staining of CD133 (green) and OXTR (red) in FFPE samples of human lung squamous carcinoma. Magnification: 40× (top) and 63× (bottom). (b) Example FACS data of gating CD133 and OXTR double positive cells in H226 and H520 cell lines. (c) Co-IF staining of CD133 and OXTR in primary lung squamous carcinoma cells, Magnification: 40×. (d) Percentage of CD133 and OXTR double positive cells in NSCLC cell lines.

FIG. 4. Tumorigenic SQCC sphere cells ubiquitously express OXTR. (a) 500 single H226 cells unsorted or enriched for OXTR, CD133, both or neither grew into cell colony in complete medium or tumor spheres in stem cell medium. Cell colony and spheres were co-immunostained for OXTR (green) and CD133 (red), counterstained with DAPI (blue). (b) Sphere size of H226 cells unsorted or enriched for OXTR, CD133, both or neither. *: p<0.05. (c) Co-immunofluorescence staining of OXTR (green) and CD133 (red) on primary SQCC spheres cultured in stem cell medium, counterstained with DAPI (blue). (d) Frequency of TICs in tumor sphere cells or regular cancer cells of H226 and H520 cell lines, and in normal lung cells of NL20 cell line.

FIG. 5. Activity of OXTR affects cell growth of TIC. (a) Proliferation of H226 and H520 cells treated with L-368,899 (L3) and oxytocin (OXT) for three days. (b) Fold change of numbers of H226 and H520 tumor spheres treated with L3 and OXT for three days. (c) Phase contrast images of tumor spheres treated with L3 or OXT. Scale bar: 150 μm. (d) Size of H226 and H520 tumor spheres treated with OXT for three days. (e) Images of colony formation of H226 and H520 cells treated with L3. (f) Colony numbers of H226 and H520 cells treated with L3. (g) Number of primary SQCC spheres treated with L3 for three days. **: p<0.01

FIG. 6. Activity of OXTR affects tumor development. (a) Percentage of OXTR mRNA upon siRNA knockdown in H226 and H520 cells. (b) Protein level of OXTR upon siRNA knockdown in H226 and H520 cells. (c) Number of H226 and H520 spheres upon siRNA knockdown. (d) Representative images of tumors developed in mice five days after subcutaneously implanted with H226 cells transfected with scrambled or OXTR siRNA. Arrows point to visible tumors. (e) Volume of tumors developed from H226 and H520 cells transfected with scrambled or OXTR siRNA. Median bar indicated. *: p<0.05, **: p<0.01.

FIG. 7. MAPK pathway mediates OXT induced OXTR signaling. (a) ERK1/2 phosphorylation upon OXT induction in CCs and TIC of H226 and H520 cell lines. (b) Proliferation of H226 and H520 cells treated with U0126. (c) Number of H226 and H520 spheres treated with OXT, U0126 or both. (d) ERK1/2 phosphorylation upon OXT and U0126 treatment in CCs and TICs of H226 and H520 cell lines.

FIG. 8. OXT stimulates lung TIC growth through autocrine/paracrine signaling. (a) Co-immunofluorescence staining of OXTR (green) and OXT (red) on tumor spheres of H226 cell line, counterstained with DAPI (blue). Scale bar: 25 μm. (b) Co-immunofluorescence staining of OXTR (green) and OXT (red) on primary SQCC spheres, counterstained with DAPI (blue). Scale bar: 50 μm. (c) Levels of OXT secreted by CCs and TICs of H226 cell line. (d) A working model of autocrine/paracrine signaling of OXT/OXTR in TICs of SQCC.

### DEFINITIONS

The terms “cancer”, “neoplasm”, “tumor”, and “carcinoma”, are used interchangeably herein to refer to cells which exhibit relatively autonomous growth, so that they exhibit an aberrant growth phenotype characterized by a significant loss of control of cell proliferation. Cells for detection or treatment in the present application include precancerous (e.g., benign), malignant, pre-metastatic, metastatic, and non-metastatic cells. Detection of cancerous cells is of particular interest. The term “normal” as used in the context of “normal cell,” is meant to refer to a cell of an untransformed phenotype or exhibiting a morphology of a non-transformed cell of the tissue type being examined. “Cancerous phenotype” refers to any of a variety of biological phenomena that are characteristic of a cancerous cell, which phenomena can vary with the type of cancer. The cancerous phenotype may be identified by abnormalities in, for example, cell growth or proliferation (e.g., uncontrolled growth or proliferation), regulation of the cell cycle, cell mobility, cell-cell interaction, or metastasis, etc.

“Non-small-cell lung carcinoma” or “NSCLC” as used herein refers to any epithelial cancer of the lung that is not a small cell lung carcinoma. An NSCLC may be a lung squamous cell carcinoma (i.e., SQCC), lung adenocarcinoma, or a large cell carcinoma.

Tumor initiating cells (TIC) (sometimes referred to in the art as cancer stem cells or CSC) are a sub-fraction of tumor cells that appear to self-renew and give rise to differentiated tumor cells, and have been implicated as participating in tumorigenesis, metastasis and drug resistance in solid cancers. TIC may be identified by TIC specific markers, such as CD133, as is described further herein.

“Biopsy” as used herein refers to any tissue sample containing cancer cells that is obtained (e.g., by excision, needle aspiration, etc.) from a subject. The biopsy may be in the form of a cell suspension, thin section (e.g., a tissue section mounted on a slide), or any other suitable form.

“Diagnosis” as used herein includes a prediction of a subject's susceptibility to a disease or disorder, determination as to whether a subject is presently affected by a disease or disorder, prognosis of a subject affected by a disease or disorder (e.g., identification of cancerous states, stages of cancer, likelihood that a patient will die from the cancer), classification of the subject's disease or disorder into a subtype of the disease or disorder (e.g., classification of a cancer as a specific type or subset), prediction of a subject's responsiveness to treatment for the disease or disorder (e.g., positive response, a negative response, no response at all to, e.g., allogeneic hematopoietic stem cell transplantation, chemotherapy, radiation therapy, antibody therapy, small molecule compound therapy) and use of therametrics (e.g., monitoring a subject's condition to provide information as to the effect or efficacy of therapy).

A “reference” as used in the context of diagnosing or identifying a sample refers to a comparison sample (e.g., positive and/or negative control, standardized beads, etc.), a predetermined value, or a value determined based on an assessment of the sample.

“Prognosis” as used herein includes a prediction of the course of disease progression and/or disease outcome, and may include the expected duration, the function, and a description of the course of the disease Examples of prognostic predictions include prognoses of long-term survival, overall survival (OS), relapse-free survival (RFS) and/or event-free survival (EFS).

By “expression level” it is meant the level of a gene product, e.g., the normalized value determined for the RNA expression level of the gene or for the expression level of a polypeptide encoded by the gene.

The terms “treatment”, “treating” and the like are used herein refers to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. In the context of cancer, the therapeutic moiety may be effective to reduce growth of the cancer and/or induce cell death in cancer cells.

By “therapeutic moiety” it is meant a polypeptide, small molecule or nucleic acid composition that confers a therapeutic activity upon a composition. A “therapeutic moiety may an agent the changes the activity of a cancer cell (e.g., via modulation of cell signaling pathways) so as to, for example, reduce cancer growth.

“Inhibitor” as used herein refers to any agent (e.g., small molecule, macromolecule, peptide, etc.) that reduces the activity of a target molecule. “Competitive inhibitor” as used herein refers to an inhibitor that reduces binding of a binding member to a target molecule, such as the binding of a ligand to a cell-surface receptor. In the context of the OXTR, a competitive inhibitor reduces binding of the oxytocin ligand to the OXTR. The competitive inhibitor may specifically bind to the active site of the enzyme (e.g., OXTR) or an allosteric site of the enzyme, or may specifically bind the substrate itself. “Non-competitive inhibitor” as used herein refers to an inhibitor that reduces activity of an inhibitor regardless of the presence of the substrate. A non-competitive inhibitor may bind to an active site of the target molecule or to an allosteric site of the target molecule.

The term “peptidomimetic” and “mimetic” and the like, refer to a modified peptide. As used herein, an “oxytocin mimetic” refers to a peptide having a similar amino acid sequence to oxytocin, with one or more amino acid substitutions, unnatural amino acids, side chain modifications, or any other suitable modification.

The terms “individual,” “subject,” “host,” and “patient,” are used interchangeably herein and refer to any mammalian subject for whom diagnosis, treatment, or therapy is desired, particularly humans.

The terms “specific binding,” “specifically binds,” and the like, refer to the preferential binding of a binding element (e.g., one binding pair member to the other binding pair member of the same binding pair) relative to other molecules or moieties in a solution or reaction mixture. The binding element may specifically bind (e.g., covalently or non-covalently) to a particular epitope or narrow range of epitopes within the cell. In certain aspects, the binding element non-covalently binds to a target.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

### DETAILED DESCRIPTION

Methods of modulating lung cancer, e.g., squamous cell carcinoma (SQCC), tumor initiating cells (TIC) are provided. Aspects of the methods including contacting a TIC with an OXTR modulatory agent, e.g., an inhibitory agent, in a manner sufficient to modulate the TIC. Aspects of the invention further include compositions that find use in practicing methods of the method. The methods and compositions find use in a variety of different applications, including but not limited to the treatment of SQCC.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g., polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Methods of Modulating Lung Tumor Initiating Cells (TIC)

As summarized above, aspects of the invention include methods of modulating lung Tumor Initiating Cells (TIC). While the lung TIC may vary, in some instances the lung TIC are lung squamous cell carcinoma (SQCC) TIC. In some instances, modulating results in reducing growth of the target TIC. By reducing growth is meant that the growth of the TIC, e.g., as measured using the sphere forming assay described in the Experimental section, below, is reduced as compared to a suitable control, where the magnitude of reduction is, in some instances, 2-fold or greater, such as 5-fold or greater, including 10-fold or greater.

Aspects of the methods include contacting the TIC with an amount of an oxytocin receptor (OXTR) modulatory agent, e.g., an OXTR inhibitory agent, effective to modulate the growth of TIC, (e.g., the proliferation of the TIC) as desired. The OXTR modulatory agent may be any suitable agent that modulates OXTR activity, OXTR-oxytocin binding, or OXTR expression, as described below.

An OXTR modulatory agent is any agent that specifically modulates OXTR signaling, signaling downstream of the OXTR or OXTR expression. Examples of OXTR modulatory agents include OXTR antagonists, such as competitive inhibitors and non-competitive inhibitors. An OXTR antagonist may prevent or reduce OXTR-oxytocin binding. Additionally or alternatively, an OXTR antagonist may reduce OXTR signaling (e.g., regardless of whether OXTR is bound to oxytocin). In certain aspects, the OXTR modulatory agent may include an oxytocin mimetic, i.e., a peptide having a similar amino acid sequence to oxytocin, one or more amino acid substitutions, unnatural amino acids, or any other suitable modification. Examples of oxytocin mimetics that act as OXTR antagonists include Atosiban and Barusiban. Atosiban is a desamino-oxytocin analogue. Barusiban is an oxytocin mimetic in which the disulfide bridge between two cysteine residues is replaced with a thioether. An oxytocin mimetic of the subject invention may be 8 or 9 amino acids in length. An oxytocin mimetic may have one or more, two or more, three or more, or four or more chemical modifications as compared to oxytocin.

In certain aspects, the OXTR modulatory agent may be a small molecule. For example, the OXTR modulatory agent may be 1 kDa or less, 900 Da or less, 800 Da or less, 700 Da or less, 600 Da or less, 500 Da or less, 400 Da or less, 300 Da or less, 200 Da or less, or 100 Da or less, where in such instances the agent may be 10 Da or more, such as 25 Da or more, including 50 Da or more. Small molecule compounds may be dissolved in water or alcohols or solvents such as DMSO or DMF, and diluted into water or an appropriate buffer prior to being provided to cells. The small molecule may be a competitive inhibitor of OXTR-oxytocin binding or a non-competitive inhibitor of OXTR activity. For example, the small molecule may be one of Atosiban, Retosiban, Epelsiban, L-368,889, L-371,257, SSR-126,768, WAY-162,720, or a derivative thereof. Atosiban is a competitive OXTR inhibitor, is also known as Tractocile, and Antocin, and may be administered by IV. Atosiban has an IUPAC name of 1-(3-mercaptopropanoic acid)-2-(O-ethyl-D-tyrosine)-4-L-threonine-8-L-ornithine-oxytocin and a Chemical Abstracts Service registry number (CAS number) of 90779-69-4. Retosiban is an orally administered OXTR agonist, and is also known as GSK-221,149. The IUPAC name of retosiban is 3R,6R)-6-[(2S)-butan-2-yl]-3-(2,3-dihydro-1H-inden-2-yl)-1-[(1R)-1-(2-methyl-1,3-oxazol-4-yl)-2-(morpholin-4-yl)-2-oxoethyl]piperazine-2,5-dione, and the CAS number is 820957-38-8. Derivatives of Retosiban may share the structural motif of Retosiban. For example, Epelsiban is a derivative of Retrosiban (also known by the name GSK-557,296-B) and has an IUPAC name of (3R,6R)-3-(2,3-dihydro-1H-inden-2-yl)-1-[(1R)-1-(2,6-dimethylpyridin-3-yl)-2-(morpholin-4-yl)-2-oxoethyl]-6-[(1S)-1-methylpropyl]piperazine-2,5-dione and a CAS number of 872599-83-2. L-368,899 is an OXTR inhibitor that may be administered orally. The IUPAC name of L-368-899 is (S)-2-amino-N-((1S,2S,4R)-7,7-dimethyl-1-((4-o-tolylpiperazin-1-lsulfonyl)methyl)bicyclo[2.2.1]heptan-2-yl)-4-(methylsulfonyl)butanamide, and the CAS number is 148927-60-0. Derivatives of L-368,899 may share the structural motif of L-368,899. L-371,257 is an OXTR inhibitor that may be administered orally, and has low permeability across the blood brain barrier (BBB). L-371,257 has an IUPAC name of 1-[4-[(1-Acetyl-4-piperidinyl)oxy]-2-methoxybenzoyl]-4-(2-oxo-2H-3,1-benzoxazin-1(4H)-yl)piperidine and a CAS number of 162042-44-6. L-372,662 is also known as L012255, and has an IUPAC name of 1-[1-[2-methoxy-4-[1-[(2-methyl-1-oxidopyridin-1-ium-3-yl)methyl]piperidin-4-yl]oxybenzoyl]piperidin-4-yl]-4H-3,1-benzoxazin-2-one.

The OXTR modulatory agent may optionally include a moiety preventing transport across the blood brain barrier (BBB). For example, an OXTR inhibitor such as L-371,257, which has low permeability across the BBB, may be used.

OXTR modulatory agents that may find use in embodiments of the invention include those described in U.S. Pat. No. 5,356,904, U.S. Pat. No. 5,464,788, U.S. Pat. No. 5,756,497, U.S. Pat. No. 5,756,504, U.S. Pat. No. 6,977,254, and US Publication No. US20070185162, the disclosures of which are incorporated herein by reference. In certain aspects, the OXTR modulatory agent may be an OXTR inhibitor, and may be a camphor sulphonamide, benzoxazinylpiperidine, pyrrolidine oxime, indolin-2-one, biaryl sulfonamide, triazole, 2,5-diketopiperzine, or any other suitable class of compounds. A review of OXTR inhibitors is provided by Borthwick et al. (J. Med. Chem. 2010, 53, 6525-38) and by Manning et al. (J. Neuroendocrinology, 2012, 24, 609-628).

In certain aspects, the OXTR modulatory agent may include an OXTR specific binding member. The terms “specific binding,” “specifically binds,” and the like, refer to the preferential binding of a domain (e.g., one binding pair member to the other binding pair member of the same binding pair) relative to other molecules or moieties in a solution or reaction mixture. The binding domain may specifically bind (e.g., covalently or non-covalently) to a particular epitope or narrow range of epitopes within the cell. In such instances, the OXTR specific binding member association with OXTR may be characterized by a KD (dissociation constant) of 10⁻⁵ M or less, 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, including 10⁻¹⁶ M or less.

A variety of different types of specific binding members may be employed. Binding members of interest include, but are not limited to, antibodies, proteins, peptides, haptens, nucleic acids, aptamers, etc. In certain aspects, the OXTR specific binding member may be an antibody or a fragment thereof. The term “antibody” as used herein includes polyclonal or monoclonal antibodies or fragments thereof that are sufficient to bind to an analyte of interest. The fragments can be, for example, monomeric Fab fragments, monomeric Fab′ fragments, or dimeric F(ab)′2 fragments. Also within the scope of the term “antibody” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies.

In certain embodiments, the agent may be an agent that modulates, e.g., inhibits, expression of functional OXTR. Inhibition of OXTR expression may be accomplished using any convenient means, including use of an agent that inhibits OXTR expression, such as, but not limited to: antisense agents, RNAi agents, agents that interfere with transcription factor binding to a promoter sequence of the OXTR gene, or inactivation of the OXTR gene, e.g., through recombinant techniques, etc.

For example, antisense molecules can be used to down-regulate expression of OXTR in the cell. The anti-sense reagent may be antisense oligodeoxynucleotides (ODN), such as synthetic ODN having chemical modifications from native nucleic acids, nucleic acid constructs that express such anti-sense molecules as RNA, and so forth. The antisense sequence may be complementary to the mRNA of the targeted protein (i.e., OXTR). Antisense molecules inhibit gene expression through various mechanisms, e.g., by reducing the amount of mRNA available for translation, through activation of RNAse H, or steric hindrance. One or a combination of antisense molecules may be administered, where a combination may include multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule may be a synthetic oligonucleotide. Antisense oligonucleotides may be at least 7 nucleotides in length, at least 10 nucleotides in length, at least 15 nucleotides in length, at least 20 nucleotides in length, 500 or fewer nucleotides in length, 100 or fewer nucleotides in length, 50 or fewer nucleotides in length, 25 or fewer nucleotides in length, between 7 and 50 nucleotides in length, between 10 and 25 nucleotides in length, and so forth, where the length is governed by efficiency of inhibition, specificity, including absence of cross-reactivity, and the like.

A specific region or regions of the endogenous sense strand mRNA sequence may be chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra, and Milligan et al., supra.) Oligonucleotides may be chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 9523225, and Beigelman et al. (1995), Nucl. Acids Res. 23:4434-42). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al. (1995), Appl. Biochem. Biotechnol. 54:43-56.

In addition, the transcription level of an OXTR can be regulated by gene silencing using RNAi agents, e.g., double-strand RNA (Sharp (1999) Genes and Development 13: 139-141). RNAi, such as double-stranded RNA interference (dsRNAi) or small interfering RNA (siRNA), has been extensively documented in the nematode C. elegans (Fire, A., et al, Nature, 391, 806-811, 1998) and routinely used to “knock down” genes in various systems. RNAi agents may be dsRNA or a transcriptional template of the interfering ribonucleic acid which can be used to produce dsRNA in a cell. In these embodiments, the transcriptional template may be a DNA that encodes the interfering ribonucleic acid. Methods and procedures associated with RNAi are also described in WO 03/010180 and WO 01/68836, all of which are incorporated herein by reference. dsRNA can be prepared according to any of a number of methods that are known in the art, including in vitro and in vivo methods, as well as by synthetic chemistry approaches. Examples of such methods include, but are not limited to, the methods described by Sadher et al. (Biochem. Int. 14:1015, 1987); by Bhattacharyya (Nature 343:484, 1990); and by Livache, et al. (U.S. Pat. No. 5,795,715), each of which is incorporated herein by reference in its entirety. Single-stranded RNA can also be produced using a combination of enzymatic and organic synthesis or by total organic synthesis. The use of synthetic chemical methods enable one to introduce desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference in its entirety). A number of options can be utilized to deliver the dsRNA into a cell or population of cells such as in a cell culture, tissue, organ or embryo. For instance, RNA can be directly introduced intracellularly. Various physical methods are generally utilized in such instances, such as administration by microinjection (see, e.g., Zernicka-Goetz, et al. (1997) Development 124:1133-1137; and Wianny, et al. (1998) Chromosoma 107: 430-439). Other options for cellular delivery include permeabilizing the cell membrane and electroporation in the presence of the dsRNA, liposome-mediated transfection, or transfection using chemicals such as calcium phosphate. A number of established gene therapy techniques can also be utilized to introduce the dsRNA into a cell. By introducing a viral construct within a viral particle, for instance, one can achieve efficient introduction of an expression construct into the cell and transcription of the RNA encoded by the construct. RNA agents that may be employed in embodiments of the invention also include miRNA agents.

In some instances, the amount of the OXTR modulatory agent that is employed is one that is effective to reduce proliferation of a SQCC TIC cell. Specifically the OXTR modulatory agent may be effective to reduce proliferation of a SQCC sub-population that expresses higher levels of OXTR, such as a TIC subset, as compared to the general lung cancer cell population. An effective amount may be 200× the calculated IC50 or less. By “IC50” is intended the concentration of a drug required for 50% inhibition in vitro. The amount (e.g., effective amount, amount to be administered, etc.) of an OXTR modulatory agent may be 200× or less, 150× or less, 100× or less, 75× or less, 60× or less, 50× or less, 45× or less, 40× or less, 35× or less, 30× or less, 25× or less, 20× or less, 15× or less, 10× or less, 8× or less, 5× or less, or 2× or less than the calculated IC50. In one embodiment, the effective amount may be 1× to 100×, 2× to 40×, 5× to 30×, or 10× to 20× of the calculated IC50.

In some instances, the amount of the OXTR modulatory agent that is employed is one that is effective to reduce lung, e.g., SQCC, TIC mediated tumorigenesis. The term tumorigenesis is employed in its conventional sense to refer to the process of initiating and promoting the development of a tumor. While the magnitude of tumorigenesis reduction, as compared to a suitable control, may vary, in some instances the magnitude of reduction is 2-fold or greater, such as 5-fold or greater, including 10-fold or greater.

In certain aspects, the TIC may be contacted with the OXTR modulatory agent in vitro. For example, the TIC may be of a SQCC cell line (e.g., H226 cells) or of a primary SQCC cell culture. In certain aspects, the step of contacting may include culturing the TIC with the OXTR modulatory agent.

Alternatively or in addition, the TIC may be contacted with the OXTR modulatory agent in vivo. In certain aspects, the step of contacting may include administering the OXTR modulatory agent to a subject having the SQCC. For example, the OXTR modulatory agent may be administered by enteric administration (e.g., oral administration) or by parenteral administration (e.g., intravenous, intra-arterial, intra-muscular or subcutaneous administration, etc.). In addition, the OXTR modulatory agent can be incorporated into a pharmaceutical composition suitable for administration to an animal subject, according to any of the embodiments discussed herein. The subject may be any suitable animal, such as a rodent (e.g. mouse, rat, etc.), primate (e.g., human, monkey, etc.), and so forth. In one embodiment, the subject may be a mouse. In another embodiment, the subject may be a human.

The step of contacting may include contacting the TIC with the OXTR modulatory agent for between 1 and 20 days, between 2 and 10 days, 1 day or more, 2 days or more, 5 days or more, 10 days or more, 20 days or more, 2 days or less, 5 days or less, 10 days or less, 20 days or less, 50 days or less, and so forth.

The SQCC of any of the above embodiments may be of any suitable animal. For example, the SQCC may be a human SQCC. Alternatively, the SQCC may be a rodent SQCC (e.g., a murine SQCC). The SQCC may be primary cells obtained from a subject having SQCC, or may be an immortalized SQCC cell line.

### Methods of Treatment

Aspects of the invention include methods of treating a subject for a lung squamous cell carcinoma (SQCC). Embodiments of the methods may include administering to the subject an amount of an oxytocin receptor (OXTR) modulatory agent effective to treat the subject for the SQCC. The OXTR modulatory agent may be any suitable agent, e.g., where the agent may be one that modulates OXTR activity, OXTR-oxytocin binding, or OXTR expression, as described above. As indicated above, treatment may be manifested in a variety of different ways. In some instances, treatment manifest by the presence of one or more of reduced tumorigenesis, metastasis and drug resistance.

The OXTR modulatory agent may be administered by any suitable route of administration, such as by enteric administration (e.g., oral) or by parenteral administration (e.g., intravenous, intra-arterial, intra-muscular, subcutaneous, etc.). In addition, the OXTR modulatory agent can be incorporated into a variety of formulations for therapeutic administration. More particularly, the agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the agent can be achieved in various ways, including oral, buccal, rectal, parenteral, intraperitoneal, intradermal, transdermal, intracheal, etc., administration. In pharmaceutical dosage forms, the agent may be administered alone or in combination with other pharmaceutically active compounds.

The OXTR modulatory agent may be administered in an amount sufficient to reduce growth (e.g., proliferation) of a SQCC TIC cell. The OXTR modulatory agent may be effective to reduce proliferation of an SQCC cell that express higher levels of OXTR, such as a TIC subset.

The OXTR modulatory agent may be administered by infusion or by local injection, e.g., by infusion at a rate of 50 mg/h to 400 mg/h, including 75 mg/h to 375 mg/h, 100 mg/h to 350 mg/h, 150 mg/h to 350 mg/h, 200 mg/h to 300 mg/h, 225 mg/h to 275 mg/h. Exemplary rates of infusion can achieve a desired therapeutic dose of, for example, 0.5 mg/m2/day to 10 mg/m2/day, including 1 mg/m2/day to 9 mg/m2/day, 2 mg/m2/day to 8 mg/m2/day, 3 mg/m2/day to 7 mg/m2/day, 4 mg/m2/day to 6 mg/m2/day, 4.5 mg/m2/day to 5.5 mg/m2/day. Administration (e.g., by infusion) can be repeated over a desired period, e.g., repeated over a period of 1 day to 5 days or once every several days, for example, five days, over 1 month, 2 months, etc. It also can be administered prior, at the time of, or after other therapeutic interventions, such as surgical intervention to remove cancerous cells.

As an example, the amount of a dose or dosing regimen sufficient to reduce growth of the SQCC (i.e., an “effective amount”) can be gauged from the IC50 of a given OXTR modulatory agent for inhibiting cell migration. By “IC50” is intended the concentration of a drug required for 50% inhibition in vitro.

With respect to the OXTR modulatory agent of the present disclosure, an effective amount may be 200× the calculated IC50 or less. The amount of a therapeutic moiety that is administered may be 200× the calculated IC50 or less. For example, the amount (e.g., effective amount, amount to be administered, etc.) of an OXTR modulatory agent may be 200× or less, 150× or less, 100× or less, 75× or less, 60× or less, 50× or less, 45× or less, 40× or less, 35× or less, 30× or less, 25× or less, 20× or less, 15× or less, 10× or less, 8× or less, 5× or less, or 2× or less than the calculated IC50. In one embodiment, the effective amount may be 1× to 100×, 2× to 40×, 5× to 30×, or 10× to 20× of the calculated IC50.

Alternatively, the effective amount can be gauged from the EC50 of a given therapeutic moiety concentration. By “EC50” is intended the plasma concentration required for obtaining 50% of a maximum effect in vivo. In related embodiments, dosage may also be determined based on ED50 (effective dosage). An effective amount may be 200× the calculated EC50 or less. The amount (e.g., effective amount, amount to be administered, etc.) of an OXTR modulatory agent may be 200× or less, 150× or less, 100× or less, 75× or less, 60× or less, 50× or less, 45× or less, 40× or less, 35× or less, 30× or less, 25× or less, 20× or less, 15× or less, 10× or less, 8× or less, 5× or less, or 2× or less than the calculated EC50. In one embodiment, the effective amount may be 1× to 100×, 2× to 40×, 5× to 30×, or 10× to 20× of the calculated EC50.

Effective amounts may readily be determined empirically from assays, from safety and escalation and dose range trials, individual clinician-patient relationships, as well as in vitro and in vivo assays such as those described in the art (e.g., Reagan-Shaw et al. (2007) The FASEB Journal 22:659-661).

In certain aspects, methods of treatment may include diagnosing the SQCC (i.e., identifying the carcinoma as a SQCC) prior to the step of contacting, based on any suitable methodology known in the art. For example, the step of diagnosing may include obtaining a biopsy of the SQCC and identifying the SQCC based on histology (e.g., based on an H&E stain of the SQCC biopsy). The SQCC may be diagnosed based on expression of OXTR, or based upon co-expression of OXTR and a CSC marker (such as CD133). For example, the method may include diagnosing the carcinoma as a SQCC when OXTR is co-expressed with the CSC marker.

The method may further include providing a prediction of whether growth (e.g., proliferation) of the TIC may be modulated by an OXTR modulatory agent, based on OXTR co-expression with a TIC marker in cells of the SQCC and prior to the step of contacting. In certain aspects, the TIC marker may be CD133. CD133 (also known as Prominin 1, or PROM1) is a glycoprotein that is expressed on certain stem cells and progenitor cells. Methods of providing a prediction of whether proliferation of the TIC may be modulated by an OXTR modulatory agent are further described in the below sections.

In certain aspects, the method of treatment includes administering the OXTR modulatory agent in addition to another suitable cancer therapy, such as surgery (e.g., surgical removal of cancerous tissue), radiation therapy, chemotherapeutic treatment, biological response modifier treatment, or a combination thereof. Such methods may be methods of simultaneously reducing tumor burden and reducing, including inhibiting, tumorigenesis.

As indicated above, the additional therapy may vary. Radiation therapy includes, but is not limited to, X-rays or gamma rays that are delivered from either an externally applied source such as a beam, or by implantation of small radioactive sources. For example, the method may further include administering an amount of a cancer chemotherapeutic agent effective to treat the subject for the SQCC. Chemotherapeutic agents are non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones. Chemotherapeutic agents are further discussed in the following sections.

Where the additional therapy comprises an additional anti-cancer active agent, e.g., a chemotherapeutic agent, the OXTR modulatory agent and the additional anti-cancer active agent are administered to the subject in combination. By “in combination” is meant that one of the agents is administered anywhere from simultaneously to up to 5 hours or more, e.g., 10 hours, 15 hours, 20 hours or more, prior to or after the other agent. In certain embodiments, the OXTR modulatory agent and the additional anti-cancer active agent are administered sequentially, e.g., where the OXTR modulatory agent is administered before or after the additional anti-cancer active agent. In yet other embodiments, the OXTR modulatory agent and the additional anti-cancer active agent are administered simultaneously, e.g., where the OXTR modulatory agent and additional anti-cancer active agent are administered at the same time as two separate formulations or are combined into a single composition that is administered to the subject, e.g., as described above in the Pharmaceutical Compositions section. Regardless of whether the two types of agents are administered sequentially or simultaneously, as illustrated above, the agents are considered to be administered together or in combination for purposes of the present invention. Routes of administration of the two agents may vary, where representative routes of administration are described in greater detail below.

The SQCC of any of the above embodiments may be of any suitable animal, such as a rodent (e.g., mouse, rat, etc.) or a primate (e.g., human, monkey, etc.). In certain aspects, the SQCC may be a human SQCC.

As described above, treatment may manifest in a number of different ways. In some instances, treatment of a subject as described herein results in an improvement in survival rate for a given condition, where the improvement may be realized in terms of an increased length of survival as compared to a control, e.g., by 1 month or longer, such as 6 months or longer, including 1 year or longer.

### Methods of Predicting Efficacy of Treatment

Aspects of the invention are directed to a method of predicting whether a non-small cell lung carcinoma (NSCLC) of a subject may be treated by modulating the oxytocin receptor (OXTR). The method may include evaluating OXTR expression by an NSCLC cell of the subject. The method may further include providing a prediction of whether an OXTR modulatory agent would be effective to treat the subject for the NSCLC based on the evaluation. The NSCLC may be a lung squamous cell carcinoma (SQCC). In certain aspects, the step of evaluating may include microscopy or flow cytometry, as described further below.

In certain aspects, the step of providing the prediction is based on a comparison of OXTR expression by the NSCLC cell to a reference. The reference may include control cells, such as a positive control cell line, such as an SQCC cell line (e.g., H226 cells), or a negative control cell line, such as an adenocarcinoma (e.g., HCC827), large cell carcinoma, an epithelial cell line, or any other suitable cell line. Alternatively, the reference may include standardized beads (such as beads conjugated to OXTR or a fragment thereof, beads providing a detectable signal, etc.). In certain aspects, the reference may be other cells from the subject (e.g., other SQCC cells, epithelial cells), which may express a lower amount of OXTR.

The method may further include evaluating expression of a TIC marker by the NSCLC cell. For example, the TIC marker may be CD133. When the expression of OXTR and the TIC marker are both evaluated, the step of providing the prediction may be based on the co-expression of OXTR and the TIC marker by the NSCLC cell.

As described above, the step of contacting may include contacting the sample with a TIC specific binding element, such as a CD133 specific binding element (e.g., a CD133 specific antibody). As such, the step of detecting may optionally further include detecting a second signal provided by a TIC specific binding element. In one example, the step of providing a prediction may be based on both the first and second signals (e.g., relating to co-expression of OXTR and CD133). For example, a subset of TIC cells may be identified a second signal obtained from a TIC specific binding member, and the step of providing a prediction may be based on the first signal detected from cells in the subset.

The step of evaluating OXTR expression may include contacting cells of the NSCLC with an oxytocin receptor (OXTR) specific binding element. The duration of the contacting step may be sufficient to allow binding of the OXTR specific binding element to OXTR (e.g., 5 or more minutes). A variety of different types of binding elements may be employed. Binding domains of interest include, but are not limited to, antibody binding agents, proteins, peptides, haptens, nucleic acids, etc. In certain aspects, the OXTR specific binding element may be an oxytocin peptide or an oxytocin mimetic, as described herein. In other aspects, the OXTR specific binding element may be an OXTR specific antibody or fragment thereof, as described herein.

In certain aspects, the OXTR specific binding element (and optionally a TIC specific binding element) may include (e.g., may be conjugated to) a detectable label. The detectable label may be a fluorescent dye, a phosphorescent dye, a colorimetric dye, or a radioactive agent. In certain aspects, the detectable label may be a fluorescent dye. The fluorescent dye may be detectable based on, for example, fluorescence emission maxima, fluorescence polarization, fluorescence lifetime, light scatter, mass, or a combination thereof.

Fluorescent dyes can be selected from any of the many dyes suitable for use in analytical applications (e.g., flow cytometry, imaging, etc.). A large number of dyes are commercially available from a variety of sources, such as, for example, Molecular Probes (Eugene, Oreg.) and Exciton (Dayton, Ohio). Examples of fluorophores that may be incorporated into the microparticles include, but are not limited to, 4-acetamido-4′-isothiocyanatostilbene-2,2′disulfonic acid; acridine and derivatives such as acridine, acridine orange, acrindine yellow, acridine red, and acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanine and derivatives such as cyanosine, Cy3, Cy5, Cy5.5, and Cy7; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylaminocoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′ dimethoxy-4′5′-dichloro-6-carboxyfluorescein (JOE), fluorescein isothiocyanate (FITC), fluorescein chlorotriazinyl, naphthofluorescein, and QFITC (XRITC); fluorescamine; IR144; IR1446; Green Fluorescent Protein (GFP); Reef Coral Fluorescent Protein (RCFP); Lissamine™; Lissamine rhodamine, Lucifer yellow; Malachite Green isothiocyanate; 4-methylumbelliferone; ortho cresolphthalein; nitrotyrosine; pararosaniline; Nile Red; Oregon Green; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), 4,7-dichlororhodamine lissamine, rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, and tetramethyl rhodamine isothiocyanate (TRITC); riboflavin; rosolic acid and terbium chelate derivatives; xanthene; or combinations thereof. Other fluorophores or combinations thereof known to those skilled in the art may also be used.

After contacting the cells with the OXTR specific binding element, the sample may be washed to remove unbound OXTR specific binding elements. Any suitable reagent may be used in washing, such as a buffer (HEPES, PBS, other, phosphate buffers, lactate buffers, etc.) or media (e.g., dMEM, HBSS, RPMI, Iscove's medium, etc.).

The step of evaluating may further include detecting a first signal provided by the OXTR specific binding element bound to the NSCLC cell. The first signal may be, for example, a fluorescence emission maxima, fluorescence polarization, fluorescence lifetime, light scatter, mass, or a combination thereof. Detecting the first signal may include quantifying the intensity of the first signal. The first signal may be indicative of the level of OXTR expression. In certain aspects, the signal may be detected on a cell-by-cell basis.

It will be understood by those of skill in the art that the stated expression levels reflect detectable amounts of the marker protein on the cell surface. A cell that is negative for staining (the level of binding of a marker specific reagent is not detectably different from an isotype matched control) may still express minor amounts of the marker. And while it is commonplace in the art to refer to cells as “positive”, “high”, etc. or “negative”, “low”, etc., for a particular marker, actual expression levels are quantitative traits. The number of molecules on the cell surface can vary by several logs, yet still be characterized as “positive”.

Any suitable protocol may be used to detect the first signal, such as fluorescence microscopy, flow cytometry, ELISA, western blotting, mass spectrometry, proteomic arrays, and so forth. In certain aspects, the staining intensity of cells can be monitored by flow cytometry, where lasers detect the quantitative levels of fluorochrome (which is proportional to the amount of cell surface marker bound by specific reagents, e.g., antibodies). Flow cytometry, or FACS, can also be used to separate cell populations based on the intensity of binding to a specific reagent, as well as other parameters such as cell size and light scatter. Although the absolute level of staining may differ with a particular fluorochrome and reagent preparation, the data can be normalized to a control.

In certain aspects, the method may include obtaining a non-small cell lung carcinoma (NSCLC) biopsy from an individual having NSCLC, prior to the step of evaluating OXTR expression. The biopsy may be obtained by excision (e.g., surgically), by needle aspiration, or by any other suitable method.

In certain aspects, the biopsy may be a cell dispersion or suspension in a solution. The solution may be a balanced salt solution, e.g., normal saline, PBS, Hank's balanced salt solution, etc., conveniently supplemented with fetal calf serum, human platelet lysate or other factors, in conjunction with an acceptable buffer at low concentration, such as from 5-25 mM. Convenient buffers include HEPES, phosphate buffers, lactate buffers, etc. The separated cells may be collected in any appropriate medium that maintains the viability of the cells. Various media are commercially available and may be used according to the nature of the cells, including dMEM, HBSS, dPBS, RPMI, Iscove's medium, etc., and may be supplemented with fetal calf serum or human platelet lysate. In other aspects, the biopsy may be a tissue section. For example, the biopsy may be a thin tissue section mounted on a microscopy slide. The biopsy of any of the above embodiments may be fixed and/or permeabilized (e.g., as described below).

The sample may be a whole sample, e.g., in crude form. Alternatively, the sample may be fractionated prior to analysis, e.g., by density gradient centrifugation, panning, magnetic bead sorting, fluorescence activated cell sorting (FACS), etc., to enrich for a cell type of interest.

In certain aspects, the method may further include fixing the cellular sample. The cells of the sample may be fixed through exposure to any of a number of cell fixing agents (i.e., fixation reagents), such as paraformaldehyde, glutaraldehyde, methanol, acetone, formalin, or any combination thereof. Other fixatives and fixation methods may be employed, as desired. Fixation time may vary, and in some instances ranges from 1 minute and 1 hour, such as 5 minutes and 30 minutes. The temperature at which fixation takes place may vary, and in some instances the temperature ranges from −30° C. to 30° C.

In addition, the method may further include permablizing cells in the biopsy may be treated with a permeabilization agent. Permeabilization may allow detectable labels which are specific for intracellular proteins, transcription factors and/or cytokines to enter the cell. Permeabilization may take place before, after, or at the same time as the fixation previously described. The cells of the sample may be permeabilized through exposure to any of a number of cell permeabilizing agents, such as methanol, acetone or a detergent (e.g., triton, NP-40, saponin, tween 20, digitonin, leucoperm, etc.), or a combination thereof. Permeabilization time may vary, and in some instances ranges from 1 minute to 1 hour, such as from 5 minutes to 30 minutes. The temperature at which permeabilization takes place may vary, and in some instances the temperature may range from 0° C. to 50° C.

The subject predictive methods may be used alone or in combination with other clinical methods for patient stratification known in the art, e.g., age, cytogenetics, the presence of certain molecular mutations, the altered expression levels of particular genes on the mRNA and/or protein levels, and so forth.

In certain aspects, providing a prediction includes generating a written report that includes the artisan's assessment of, for example, whether an NSCLC of a subject may be treated by modulating the OXTR. Thus, a subject method may further include a step of generating or outputting a report providing the prediction, which report can be provided in the form of an electronic medium (e.g., an electronic display on a computer monitor), or in the form of a tangible medium (e.g., a report printed on paper or other tangible medium).

In certain aspects, the method may include identification of NSCLC cell OXTR expression and optionally a TIC marker expression on the mRNA level, e.g., instead of expression on the protein level. In such aspects, the step of providing the prediction may be based on the mRNA expression rather than the protein level expression of OXTR (and optionally further a TIC marker).

A number of exemplary methods are also known in the art for measuring mRNA expression levels in a sample, include, without limitation, hybridization-based methods, e.g., northern blotting and in situ hybridization (Parker & Barnes, Methods in Molecular Biology 106:247-283 (1999)), RNAse protection assays (Hod, Biotechniques 13:852-854 (1992)), and PCR-based methods (e.g., reverse transcription PCR (RT-PCR) (Weis et al., Trends in Genetics 8:263-264 (1992)).

For measuring mRNA levels, the starting material may be total RNA or poly A+ RNA isolated from a suspension of cells, e.g., a peripheral blood sample a bone marrow sample, etc., or from a homogenized tissue, e.g., a homogenized biopsy sample, a homogenized paraffin- or OCT-embedded sample, etc. General methods for mRNA extraction are well known in the art and are disclosed in standard textbooks of molecular biology, including Ausubel et al., Current Protocols of Molecular Biology, John Wiley and Sons (1997). RNA isolation can also be performed using a purification kit, buffer set and protease from commercial manufacturers, according to the manufacturer's instructions. For example, RNA from cell suspensions can be isolated using Qiagen RNeasy mini-columns, and RNA from cell suspensions or homogenized tissue samples can be isolated using the TRIzol reagent-based kits (Invitrogen), MasterPure™ Complete DNA and RNA Purification Kit (EPICENTRE™, Madison, Wis.), Paraffin Block RNA Isolation Kit (Ambion, Inc.) or RNA Stat-60 kit (Tel-Test).

A variety of different manners of measuring mRNA levels are known in the art, e.g., as employed in the field of differential gene expression analysis. One representative and convenient type of protocol for measuring mRNA levels is array-based gene expression profiling. Such protocols are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

In specific hybridization technology practiced to generate the expression profiles, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The resultant pattern of hybridized nucleic acid provides information regarding expression for each of the genes that have been probed, where the expression information is in terms of whether or not the gene is expressed and at what level, where the expression data, i.e., expression profile (e.g., in the form of a transcriptosome), may be both qualitative and quantitative.

Alternatively, non-array based methods for quantitating the level of one or more nucleic acids in a sample may be employed. These include those based on amplification protocols, e.g., Polymerase Chain Reaction (PCR)-based assays, including quantitative PCR, reverse-transcription PCR (RT-PCR), real-time PCR, and the like, e.g., TaqMan® RT-PCR, MassARRAY® System, BeadArray® technology, and Luminex technology; and those that rely upon hybridization of probes to filters, e.g., Northern blotting and in situ hybridization.

### In Vivo Method of Visualization

Aspects of the invention are directed to an in vivo method for visualizing a lung squamous cell carcinoma (SQCC) in a subject. The method may include contacting the SQCC with an oxytocin receptor (OXTR) specific binding member conjugated to a first detectable label. The method may further include detecting a first signal provided by the first detectable label.

The OXTR specific binding member may be selected from oxytocin, an oxytocin mimetic, an OXTR-specific antibody or a fragment thereof (e.g., in accordance with any of the embodiments described herein). In addition, the first detectable label may be selected from a fluorescent dye, a phosphorescent dye, a colorimetric dye, and a radioactive agent (e.g., in accordance with any of the embodiments described herein). In one embodiment, the first signal may be detected by exposing the SQCC to a light source, such as a UV light source.

In certain aspects, the step of contacting may include topically administering the OXTR specific binding member (e.g., in a liquid or aerosol form) and optionally performing one or more wash steps (i.e., using a suitable buffer) to remove unbound OXTR specific binding members. Alternatively, the step of contacting may include enteric or parenteral administration of the OXTR specific binding member to the subject.

The method may further include contacting the SQCC with a CSC specific binding member conjugated to a second detectable label, such as a CD133 specific binding member (e.g., a CD133 specific antibody). The method may then additionally include detecting a second signal provided by the second detectable label.

In certain aspects, the subject may be any suitable animal, such as a rodent, primate, or the like. In certain aspects, the subject is a human. In certain aspects, the step of contacting may be performed prior to surgical excision of the SQCC. The excision may be targeted to tissue expressing higher levels of OXTR, e.g., as identified based on a signal provided by the detectable label.

In vivo visualization of OXTR expression of an SQCC may be performed according to the above embodiments prior to surgically excising the SQCC or a portion of the SQCC thereof. For example, the portion of the SQCC that expresses a higher level of OXTR (e.g., a TIC enriched portion) may be preferentially excised. In vivo visualization of OXTR expression of an SQCC in an animal model may find use in cancer research.

### Methods of Screening

Aspects of the invention are directed to a method of screening an oxytocin receptor (OXTR) modulatory agent effective to treat a subject for a lung squamous cell carcinoma (SQCC). The method may include contacting SQCC cells with a potential OXTR modulatory agent. The method may further include evaluating proliferation of the SQCC cells. The SQCC cells may include TIC. For example, the SQCC cells may include CSCs that co-express OXTR and CD133.

The step of evaluating proliferation of the SQCC cells may be performed in vitro. For example, the method may include contacting the SQCC cells with a proliferation assay dye prior to culturing the SQCC cells in vitro. The proliferation assay dye may be selected from bromodeoxyuridine (BrdU), tetrazolium dye (XTT), eFluor 670 or eFluor 450 (e.g., provided by eBioscience), CellTrace (provided by Life Technologies), or any other suitable intracellular dye. Working concentrations of the above dyes are known in the art and provided by the supplier. The method may further include culturing the SQCC cells for between 1 and 20 days, between 2 and 10 days, 12 hours or more, 1 day or more, 2 days or more, 5 days or more, 10 days or more, 20 days or more, 2 days or less, 5 days or less, 10 days or less, 20 days or less, 50 days or less, and so forth in the presence of the potential OXTR modulatory agent. The step of evaluating may include measuring the proliferation assay dye (e.g., by microscopy or flow cytometry) in the SQCC cells to assess the extent of proliferation. Cells that proliferated (underwent cell division) would show a corresponding decrease in proliferation assay dye content.

In another example, the step of evaluating may include performing a sphere assay. In a sphere assay, cells are cultured under sphere-forming conditions (e.g., cultured in suspension, in hanging droplets, in round bottom wells, etc.). Following sphere formation, cells may be cultured (e.g., in suspension), for a total culture time of for example, between 1 and 20 days, between 2 and 10 days, 12 hours or more, 1 day or more, 2 days or more, 5 days or more, 10 days or more, 20 days or more, 2 days or less, 5 days or less, 10 days or less, 20 days or less, 50 days or less, and so forth in the presence of the OXTR modulatory agent. The number of spheres (e.g., total number, fold change) and/or the size (e.g., diameter) may then be measured to evaluate cell proliferation.

In another example, the SQCC cells may be cultured (e.g., for any of the above time ranges) and the optical density may be measured (e.g., at 570-630 nm) to evaluate cell proliferation. Other proliferation assays known in the art are within the scope of the embodiments described herein.

The step of evaluating may include comparing the proliferation of the SQCC cells contacted with the potential OXTR modulatory agent to SQCC cells that were untreated. A decrease in the proliferation of SQCC cells contacted with the potential OXTR modulatory agent would indicate that the agent may have a therapeutic effect. In certain aspects, the SQCC cells (those contacted with the potential OXTR modulatory agent and those untreated) may be contacted with oxytocin. The concentration of the OXTR modulatory agent (and optionally oxytocin) in the culture may be between 0.01× and 1000× the IC50, between 0.1× and 100× the IC50, or between 1× and 10× the IC50.

In certain aspects, the step of contacting may include administering the potential OXTR modulatory agent to an animal model having a lung SQCC. The step of evaluating may then include comparing the growth of the lung SQCC in the animal model treated with the potential OXTR modulatory agent to a control, such as an untreated animal model having a lung SQCC. The animal model may be a murine model, such as those described by You et al. (Cancer Metastasis Rev. 2013; 32(1-2):77-82). In one embodiment, the SQCC cells may be human SQCC cells (e.g., in a murine SQCC model).

Potential OXTR modulatory agents for screening include known and unknown compounds that encompass numerous chemical classes. Potential OXTR modulatory agents are also found among biomolecules, including peptides, oxytocin mimetics, polynucleotides, saccharides, fatty acids, steroids, purines, pyrimidines, derivatives, structural analogs or combinations thereof. Included are pharmacologically active drugs, genetically active molecules, etc. Potential OXTR modulatory agents include camphor sulphonamides, benzoxazinylpiperidines, pyrrolidine oximes, indolin-2-ones, biaryl sulfonamides, triazoles, 2,5-diketopiperzines, or any other suitable class of compounds. Compounds also include small molecules such as atosiban, retosiban, Epelsiban, L-368,889, L-371,257, SSR-126,768, WAY-162,720, or a derivative thereof. In certain aspects, the potential OXTR modulatory agent may include one of the motifs shown in FIGS. 12 to 14.

Potential OXTR modulatory agents for screening also include nucleic acids, for example, nucleic acids that encode siRNA, shRNA, antisense molecules, or miRNA. Many vectors useful for transferring nucleic acids into target cells are available. The vectors may be maintained episomally, e.g., as plasmids, minicircle DNAs, virus-derived vectors such cytomegalovirus, adenovirus, etc., or they may be integrated into the target cell genome, through homologous recombination or random integration, e.g., retrovirus derived vectors such as MMLV, HIV-1, ALV, etc. Vectors may be provided directly to the subject cells. In other words, the pluripotent cells are contacted with vectors comprising the nucleic acid such that the vectors are taken up by the cells.

Methods for contacting cells with nucleic acid vectors, such as electroporation, calcium chloride transfection, and lipofection, are well known in the art. Alternatively, the nucleic acid of interest may be provided to the subject cells via a virus. In other words, the cells may be contacted with viral particles (e.g., retroviruses, lentiviruses, etc.) comprising the nucleic acid of interest. Retroviruses, for example, lentiviruses, are particularly suitable to the method of the invention. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g., MMLV, are capable of infecting most murine and rat cell types, and are generated by using ecotropic packaging cell lines such as BOSC23 (Pear et al. (1993) P.N.A.S. 90:8392-8396). Retroviruses bearing amphotropic envelope protein, e.g., 4070A (Danos et al, supra.), are capable of infecting most mammalian cell types, including human, dog and mouse, and are generated by using amphotropic packaging cell lines such as PA12 (Miller et al. (1985) Mol. Cell. Biol. 5:431-437); PA317 (Miller et al. (1986) Mol. Cell. Biol. 6:2895-2902); GRIP (Danos et al. (1988) PNAS 85:6460-6464). Retroviruses packaged with xenotropic envelope protein, e.g., AKR env, are capable of infecting most mammalian cell types, except murine cells. The appropriate packaging cell line may be used to ensure that the subject CD33+ differentiated somatic cells are targeted by the packaged viral particles. Methods of introducing the retroviral vectors comprising the nucleic acid encoding the reprogramming factors into packaging cell lines and of collecting the viral particles that are generated by the packaging lines are well known in the art.

Vectors used for providing nucleic acid to the subject cells may comprise suitable promoters for driving the expression, that is, transcriptional activation, of the nucleic acid of interest. This may include ubiquitously acting promoters, for example, the CMV-b-actin promoter, or inducible promoters, such as promoters that are active in particular cell populations or that respond to the presence of drugs such as tetracycline. By transcriptional activation, it is intended that transcription will be increased above basal levels in the target cell by at least about 10 fold, by at least about 100 fold, by at least about 1000 fold, and so forth.

Potential OXTR modulatory agents for screening also include polypeptides. Such polypeptides may optionally be fused to a polypeptide domain that increases solubility of the product. The domain may be linked to the polypeptide through a defined protease cleavage site, e.g., a TEV sequence, which is cleaved by TEV protease. The linker may also include one or more flexible sequences, e.g., from 1 to 10 glycine residues. In some embodiments, the cleavage of the fusion protein is performed in a buffer that maintains solubility of the product, e.g., in the presence of from 0.5 to 2 M urea, in the presence of polypeptides and/or polynucleotides that increase solubility, and the like. Domains include endosomolytic domains, e.g., influenza HA domain; and other polypeptides that aid in production, e.g., IF2 domain, GST domain, GRPE domain, and the like.

The candidate polypeptide agent may be produced from eukaryotic produced by prokaryotic cells, it may be further processed by unfolding, e.g., heat denaturation, DTT reduction, etc. and may be further refolded, using methods known in the art. Modifications that do not alter primary sequence include chemical derivatization of polypeptides, e.g., acylation, acetylation, carboxylation, amidation, etc. Also included are modifications of glycosylation, e.g., those made by modifying the glycosylation patterns of a polypeptide during its synthesis and processing or in further processing steps; e.g., by exposing the polypeptide to enzymes which affect glycosylation, such as mammalian glycosylating or deglycosylating enzymes. Also embraced are sequences that have phosphorylated amino acid residues, e.g., phosphotyrosine, phosphoserine, or phosphothreonine. The polypeptides may have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. D-amino acids may be substituted for some or all of the amino acid residues.

The candidate polypeptide agent may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like. Alternatively, the candidate polypeptide agent may be isolated and purified in accordance with conventional methods of recombinant synthesis. A lysate may be prepared of the expression host and the lysate purified using HPLC, exclusion chromatography, gel electrophoresis, affinity chromatography, or other purification technique. The compositions which are used may comprise at least 20% by weight of the desired product (e.g., at least about 75% by weight, at least about 95% by weight, at least about 99.5% by weight) in relation to contaminants related to the method of preparation of the product and its purification.

In some cases, the candidate polypeptide agents to be screened are antibodies. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The specific or selective fit of a given structure and its specific epitope is sometimes referred to as a “lock and key” fit. The archetypal antibody molecule is the immunoglobulin, and all types of immunoglobulins, IgG, IgM, IgA, IgE, IgD, etc., from all sources, e.g., human, rodent, rabbit, cow, sheep, pig, dog, other mammal, chicken, other avians, etc., are considered to be “antibodies.” Antibodies utilized in the present invention may be either polyclonal antibodies or monoclonal antibodies. Antibodies may be provided in the media in which the cells are cultured.

Potential OXTR modulatory agents may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Potential OXTR modulatory agents are screened for biological activity by adding the agent to at least one or more cell samples, e.g., in conjunction with cells not contacted with the agent. The change in parameters in response to the agent is measured, and the result evaluated by comparison to reference cultures, e.g., in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agent concentrations to obtain a differential response to the various concentrations. As known in the art, determining the effective concentration of an agent may use a range of concentrations resulting from 1:10, or other log scale, dilutions. The concentrations may be further refined with a second series of dilutions, if necessary. One of these concentrations may serve as a negative control, i.e. at zero concentration or below the level of detection of the agent or at or below the concentration of agent that does not give a detectable change in the phenotype.

### Pharmaceutical Compositions

Aspects of the invention are directed to a pharmaceutical composition for the treatment of a lung squamous cell carcinoma (SQCC) in a subject. The pharmaceutical composition may include an oxytocin receptor (OXTR) modulatory agent and an additional anti-cancer active agent, e.g., cancer chemotherapeutic agent, such as an active agent known to treat lung squamous cell carcinoma (SQCC), e.g., to reduce tumor burden and inhibit tumorigenesis. The OXTR modulatory agent may be any suitable agent that modulates OXTR activity, OXTR-oxytocin binding, or OXTR expression, such as described above.

The chemotherapeutic agent may be any agent that exhibits desired activity againts lung SQCC. In certain aspects, the chemotherapeutic agent may include a non-proteinaceous compound that reduces proliferation of cancer cells. Alternatively or in addition, the chemotherapeutic agent may include a cytotoxic agent. Examples of cytotoxic chemotherapeutic agents include DNA alkylating agents and antimetabolites. The chemotherapeutic agent may be selected from Cisplatin, Carboplatin, Paclitaxel, Albumin-bound paclitaxel, Docetaxel, Gemcitabine, Vinorelbine, Irinotecan, Etoposide, Vinblastine and Pemetrexed.

Chemotherapeutic agents may be non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (CYTOXAN™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (TAXOL®), docetaxel (TAXOTERE®), deoxycoformycin, mitomycin-C, L-asparaginase, azathioprine; brequinar; alkaloids, e.g., vincristine, vinblastine, vinorelbine, vindesine, etc.; podophyllotoxins, e.g., etoposide, teniposide, etc.; antibiotics, e.g., anthracycline, daunorubicin hydrochloride (daunomycin, rubidomycin, cerubidine), idarubicin, doxorubicin, epirubicin and morpholino derivatives, etc.; phenoxizone biscyclopeptides, e.g., dactinomycin; basic glycopeptides, e.g., bleomycin; anthraquinone glycosides, e.g., plicamycin (mithramycin); anthracenediones, e.g., mitoxantrone; azirinopyrrolo indolediones, e.g., mitomycin; macrocyclic immunosuppressants, e.g., cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (TAXOL®), TAXOL® derivatives, docetaxel (TAXOTERE®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g., prednisone, dexamethasone, etc.; estrogens and pregestins, e.g., hydroxyprogesterone caproate, medroxyprogesterone acetate, megestrol acetate, estradiol, clomiphene, tamoxifen; etc.; and adrenocortical suppressants, e.g., aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and ZOLADEX®. Estrogens stimulate proliferation and differentiation, therefore compounds that bind to the estrogen receptor are used to block this activity. Corticosteroids may inhibit T cell proliferation.

Other chemotherapeutic agents include metal complexes, e.g., cisplatin (cis-DDP), carboplatin, etc.; ureas, e.g., hydroxyurea; and hydrazines, e.g., N-methylhydrazine; epidophyllotoxin; a topoisomerase inhibitor; procarbazine; mitoxantrone; leucovorin; tegafur; etc. Other anti-proliferative agents of interest include immunosuppressants, e.g., mycophenolic acid, thalidomide, desoxyspergualin, azasporine, leflunomide, mizoribine, azaspirane (SKF 105685); IRESSA® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL, TAXOTERE (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art.

Paclitaxel should be understood to refer to not only the common chemically available form of paclitaxel, but analogs and derivatives (e.g., TAXOTERE™ docetaxel, as noted above) and paclitaxel conjugates (e.g., paclitaxel-PEG, paclitaxel-dextran, or paclitaxel-xylose).

Chemotherapeutic agents other than those that promote cell death include agents that alter the activity of a cell. Such chemotherapeutic agents include, but are not limited to, cytokines, chemokines, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Additionally or alternatively, the chemotherapeutic agent may be myristoylated or fused to a polypeptide permeant domain to promote uptake by the cell. A number of permeant domains are known in the art and may be used in the non-integrating polypeptides of the present invention, including peptides, peptidomimetics, and non-peptide carriers. For example, a permeant peptide may be derived from the third alpha helix of Drosophila melanogaster transcription factor Antennapaedia, referred to as penetratin. As another example, the permeant peptide includes the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002). The site at which the fusion is made may be selected in order to optimize the biological activity, secretion or binding characteristics of the polypeptide. The optimal site will be determined by routine experimentation.

One or both of the OXTR modulatory agent and the chemotherapeutic agent may have a moiety that targets a cell for antibody-dependent cell-mediated cytotoxicity (ADCC) or complement dependent cytotoxicity (CDC)-dependent death, e.g., the Fc component of immunoglobulin. Moieties that promote cell death also include moieties that target a cell for antibody-dependent cell-mediated cytotoxicity (ADCC), antibody dependent cell-mediated phagocytosis (ADCP), or complement dependent cytotoxicity (CDC, also known as complement-mediated cytolysis, or CMC), e.g., the Fc component of immunoglobulin. See, for example, Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290). To assess ADCC activity of a molecule of interest, an in vitro ADCC assay may be performed. Useful effector cells for such assays include peripheral blood mononuclear cells (PBMC) and Natural Killer (NK) cells. Alternatively or additionally, ADCC activity of the molecule of interest may be assessed in vivo, e.g., in an animal model such as that disclosed in Clynes et al. PNAS (USA) 95:652-656 (1998). All FcγRs bind the same region on Fc, at the N-terminal end of the Cγ2 domain and the preceding hinge, which region may be utilized as a functional moiety for the purposes of the invention. An overlapping but separate site on Fc serves as the interface for the complement protein C1q. In the same way that Fc/FcγR binding mediates ADCC and ADCP, Fc/C1q binding mediates complement dependent cytotoxicity (CDC).

In certain aspects, the OXTR modulatory agent may be an OXTR specific binding member conjugated to the chemotherapeutic agent. chemotherapeutic agents may be bound to OXTR specific binding element of the subject compositions by covalent interactions. In some embodiments, a linker may be used, where the linker may be any moiety that can be used to link the OXTR polypeptide to the functional moiety. In some embodiments, the linker may be a cleavable linker. The use of a cleavable linker enables the moiety linked to the OXTR specific binding element to be released from the OXTR specific binding element once absorbed by the cell, and transported to the cell body. The cleavable linker may be cleavable by a chemical agent, by an enzyme, due to a pH change, or by being exposed to energy. Examples of forms of energy that may be used include light, microwave, ultrasound, and radiofrequency. In certain applications, it may be desirable to release the functional moiety, particularly where the moiety is a therapeutic moiety, once the compound has entered the cell, resulting in a release of the moiety. Accordingly, in one variation, the linker L may be a cleavable linker.

Techniques for conjugating chemotherapeutic agents to binding elements, e.g., a OXTR specific binding element, are well known in the art, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

The pharmaceutical compositions described above are compositions that include an OXTR modulatory agent and a therapeutic moiety present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g., liposomes, e.g., liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semi-solid, liquid or gaseous forms, such as tablets, capsules, powders, granules, ointments, solutions, suppositories, injections, inhalants, gels, microspheres, and aerosols. As such, administration of the therapeutic moiety can be achieved in various ways, including transdermal, intradermal, oral, buccal, rectal, parenteral, intraperitoneal, intradermal, intracheal, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

The pharmaceutical composition may further include a pharmaceutically acceptable carrier. The OXTR modulating agent and the chemotherapeutic agent may be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, the pharmaceutical composition may be suitable for administration in various ways, including oral, buccal, rectal, parenteral, intraperitoneal, intradermal, transdermal, intracheal, etc., administration.

Preparations of the pharmaceutical composition, may be sterile. Sterility is readily accomplished by filtration through sterile filtration membranes (e.g., 0.2 μm membranes). Therapeutic compositions may be placed into a container having a sterile access port, for example, an intravenous solution bag or vial having a stopper pierceable by a hypodermic injection needle. The OXTR based therapies may be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The infusion solution may be prepared by reconstituting the lyophilized compound using bacteriostatic Water-for-Injection. Alternatively, the therapeutic moiety may be formulated into lotions for topical administration.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent may be selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990). The components used to formulate the pharmaceutical compositions may be of high purity and are substantially free of potentially harmful contaminants (e.g., at least National Food (NF) grade, at least analytical grade, at least pharmaceutical grade). Moreover, compositions intended for in vivo use may be sterile.

The pharmaceutical composition can be incorporated into a variety of formulations. More particularly, the therapeutic moiety may be formulated into pharmaceutical compositions by combination with appropriate pharmaceutically acceptable carriers or diluents.

### Kits

Aspects of the invention are directed to a kit including an oxytocin receptor (OXTR) specific binding member and a TIC specific binding member. The OXTR specific binding member may be conjugated to a first detectable label. In addition, the TIC specific binding member may be conjugated to a second detectable label. As described above, the OXTR specific binding member may be oxytocin, an oxytocin mimetic, an OXTR antibody or a fragment thereof. In certain aspects the TIC specific binding member may be a CD133 specific binding member (e.g., such as a CD133 specific antibody).

One or both of the first and second detectable labels may be selected from a fluorescent dye, a phosphorescent dye, a colorimetric dye, and a radioactive agent (e.g., according to any of the embodiments described herein).

The kit may include additional binding members that specifically bind additional cellular markers. Specific binding members (e.g., OXTR specific binding member, TIC specific binding member, and any additional binding members) may be provided in separate containers or mixed in the same container.

The kit may also include one or more cell fixing reagents such as paraformaldehyde, glutaraldehyde, methanol, acetone, formalin, or any combinations or buffers thereof. Further, the kit may include a cell permeabilizing reagent, such as methanol, acetone or a detergent (e.g., triton, NP-40, saponin, tween 20, digitonin, leucoperm, or any combinations or buffers thereof. Other protein transport inhibitors, cell fixing reagents and cell permeabilizing reagents familiar to the skilled artisan are within the scope of the subject kits.

The kit may further include reagents for performing a flow cytometric assay. Examples of said reagents include buffers for at least one of reconstitution and dilution of the first and second detectable molecules, buffers for contacting a cell sample with one or both of the first and second detectable molecules, wash buffers, control cells, control beads, fluorescent beads for flow cytometer calibration and combinations thereof.

The detectable labels and/or reagents described above may be provided in liquid or dry (e.g., lyophilized) form. Any of the above components (detectable labels and/or reagents) may be present in separate containers (e.g., separate tubes, bottles, or wells in a multi-well strip or plate). In addition, one or more components may be combined into a single container, e.g., a glass or plastic vial, tube or bottle.

In certain aspects, the kit may include one or more standardized controls. The standardized controls may be control particles such as control beads or control cells.

In addition to the above components, the subject kits may further include instructions for practicing the subject methods. These instructions may be present in the subject kits in a variety of forms, one or more of which may be present in the kit. One form in which these instructions may be present as printed information on a suitable medium or substrate, e.g., a piece or pieces of paper on which the information is printed, in the packaging of the kit, in a package insert, etc. Yet another means would be a computer readable medium, e.g., diskette, CD, DVD, portable flash drive, etc., on which the information has been recorded. Yet another means that may be present is a website address which may be used via the internet to access the information at a removed site.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Experimental

### I. Materials and Methods

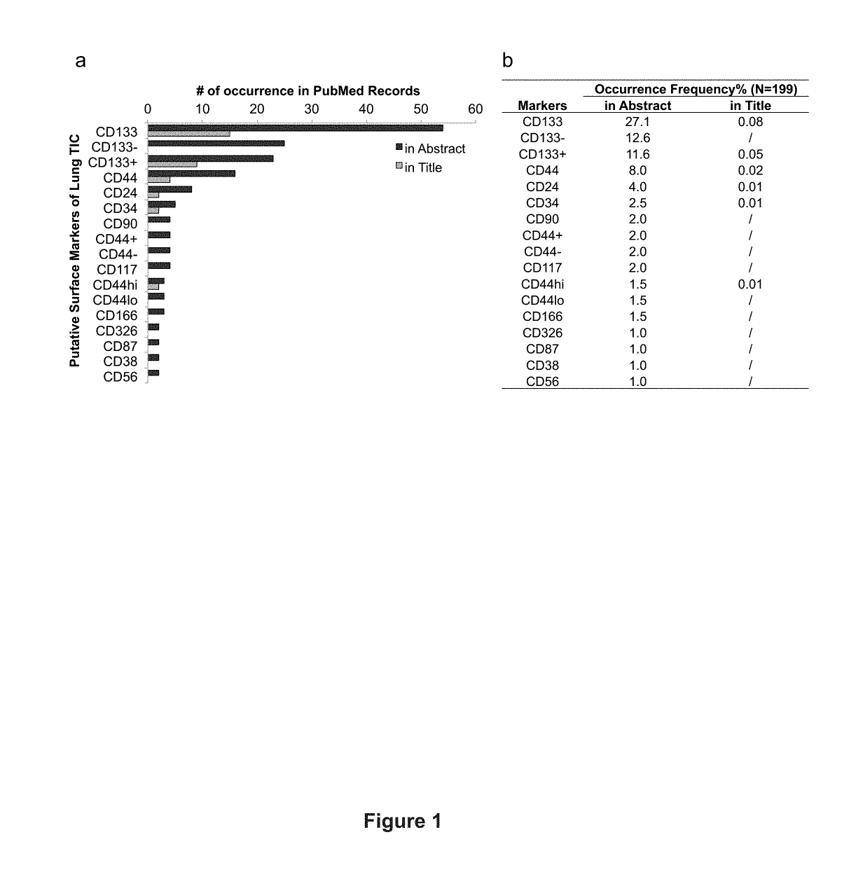

All gene expression and survival data were obtained from publicly-available resources: NCI TCGA and NCBI GEO. Human lung cancer tissue samples were purchased from US Biomax. Cell imaging was acquired using a confocal laser scanning microscope at the Stanford Fluorescence Microscopy Core. FACS was done at the Stanford FACS core facility. All sphere-forming assays and mouse xenograft model were performed at the Stanford Transgenic, Knockout and Tumor Model Center. qRT-PCR and sequencing was performed at Stanford Protein and Nucleic Acid Facility.
A. Text mining of lung TIC associated literature. PubMed literatures were queried on Apr. 13, 2015, using the search term, (“Neoplastic Stem Cells”[majr] OR “tumor-propagating cells”[All Fields]) AND “lung cancer”[All Fields]. This resulted in total 199 articles. Words with a prefix “CD” followed by digits were extracted from the title and abstract of each article. Resulted terms were manually examined. Immune cell CD markers were excluded from the list. CD marker with positive sign, such as CD133+, was treated as different from CD133 or CD133−. The number of unique occurrence of each term is counted in titles and abstracts. Terms occurred in at least two different articles were selected for ranking by its occurrence frequency.
B. Primary Tumor Cell Preparation. SQCC samples were collected from patients according to protocols approved by the Stanford IRB board. Samples were washed, dissociated, and incubated in DNase and collagenase/dispase. After incubation, cell clusters and red blood cells were removed. The single cells were resuspended and prepared for cell culture and staining.
C. Microarray datasets. The TOGA lung cancer gene expression datasets were downloaded from the TOGA data portal on September 2012. No significant changes in available sample size were found during a recent check. Twelve independent gene expression datasets of SQCC were obtained from GEO, with accession numbers listed in FIG. 1c. These datasets were selected based on two criteria: explicit pathological diagnosis of SQCC and associated with an actual publication. Pearson or Spearman correlation analysis was used for the association study, depending on whether the distribution of the gene expression data is parametric or non-parametric. Multiple hypothesis testing was corrected using false discovery rates to select associations with a q value less than 0.1. Meta-analysis of correlations were performed using R package meta, where fixed and random effects estimates were calculated and inverse variance weighting was used for pooling. Survival data was extracted from all the datasets whenever they were available. Proportional hazards regression analysis was performed with the gene expression of OXTR and CD133 as covariates. Proportional hazard hypothesis was test based on weighted residuals.
D. Cell Culture and Imaging. H226, H520, HCC827, H522 and NL20 cell lines were purchased from ATCC and cultured in recommended cell medium for bulk cancer cell growth. HCC95 and H157 were from Dr. Sage's laboratory. APC conjugated mouse anti-CD133 Ab (BioLegend 14208), goat anti-OXTR Ab (Life Technologies ab87312) with anti-goat Alexa488 (Life Technologies A11055), rabbit anti-CD44 (Santa Cruz Biotech sc-7946) with anti-rabbit Alexa594 (Life Technologies A21207) and rabbit anti-OXT with anti-rabbit Alexa594 were used to stain CD133, OXTR, CD44 and OXT respectively in human tissue samples and cell lines for immunofluorescence (IF) imaging. Goat F(ab′)₂ IgG (Life Technologies 11301C) and isotype control for rabbit primary Ab (Life Technology 08-6199) were used as isotype control of OXTR Ab and CD133 Ab. Quantitative IF imaging signal of human lung tissue samples were measured using the Bioquant image analysis software in the Nikon 8000 microscope and Leica DMI 6000B.
E. Flow Cytometry. Cells were stained with CD133, CD44 or OXTR Ab separately for single channel counting, and stained with CD133 and OXTR Ab for double channel counting and sorting. Cells were also stained for secondary Ab alone for subtraction of non-specific binding. Cells were analyzed and sorted after gating on singlet, viable DAPI-cells. For double positive population sorting, CD133 was gated first then OXTR was gated.
F. Sphere-forming assays. Clonal density of H226 and H520 cells, 1000 cells/ml, were seeded in low-attachment 6-well plate (Sigma-Aldrich, CLS3471) with serum free medium supplemented with EGF (Invitrogen, PHG0311L), bFGF (Invitrogen, PHG0024), and ITS (BD Bioscience, 354351). No other hormone factors were present in the medium. Sphere sizes were measured by the length of an ellipse model using Bioquant image analysis software. Sphere number was counted under microscope manually using a size criteria set experimentally. L-368,899 (Sigma-Aldrich, L2540) and Oxytocin (Sigma-Aldrich, O3251) were added to sphere forming medium right after seeding and administered daily for three to five days. U0126 (Cell Signaling, 9903) was added to spheres five days after seeding for 2 hrs, then spheres were collected for western blot analysis.
G. Western Blotting. Total cell lysates were collected using 1× cell lysis buffer (Cell Signaling), as directed by the manufacturer. The protein concentration was determined using the BioRad DC protein assay. SDS/PAGE analysis of 50 μg of protein was transferred to an Immobilon-P membrane (Millipore). Anti-OXTR monoclonal Ab (Abcam, ab181077) and anti-OXT antibody (Millipore, AB911) were used to detect OXTR and OXT proteins, respectively, as directed by the manufacturer. Anti-phospho-p44/42 MAPK (ERK1/2)(Thr202/Tyr204) Antibody (Cell signaling, 9101) and anti-p44/42 MAPK (ERK1/2) antibody (Cell Signaling, 9102) were used to detect the phospho ERK1/2 and ERK1/2 protein, respectively, as directed by the manufacturer.
H. Detection of OXT in cell culture. One thousand H226 cells were seeded in either stem cell medium or complete medium and cultured for 7 days. The medium was collected from both attached cancer cell culture and sphere culture. The OXT concentration in the culture medium and medium alone was measured using the Oxytocin ELISA Kit (Abcam, ab133050).
I. Transient knockdown of OXTR. OXTR siRNA (Life Technologies, AM16708 (1766)) or Scrambled siRNA (Life Technologies, AM4621) was transfected into H226 and H520 cells using the RNAiMAX kit (Life Technologies, 13778-075). Three days after transfection, 1000 cells were collected and seeded in low-attachment 6-well plate for the sphere-forming assay. The rest cells were split in half for qRT-PCR and western blot analysis of OXTR knockdown.
J. Xenotransplantation assays Healthy female and male NU/NU mice age 8 to 12 weeks were used (Charles River). Animal handling was performed in accordance with Stanford University Animal Research Committee guidelines. Four million H226 cells or two million H520 cells transfected with scrambled or OXTR siRNA were subcutaneously implanted in mice. Tumor size was measured with a digital caliper and tumor volume was calculated using an ellipsoid model with formulation π/6*(length)*(width)².
K. Data Analysis. Wilcoxon rank-sum test was used to compare treated group with non-treated group. p<0.05 was used as cutoff point for statistical significance in experiments.

### II. Results

### A. Selection of Seed Markers of Lung TIC for GBA Analysis

To choose the best surface markers as seeds for a function-specific association analysis, we applied a text-mining approach to seek the most commonly used surface markers of lung TICs. We collected all literature in PubMed that discussed neoplastic stem cells (the MeSH term of “tumor initiating cells”) as one of their major topics. Among that literature, we selected those publications that also mentioned lung cancer, which resulted in 199 publications discussing neoplastic stem cells and lung cancer. To identify the surface markers used in the title and abstract of these studies, we extracted all words in these 199 abstracts with a prefix “CD” followed by digits, provided that most of the TIC surface markers are commonly referred to by their Clusters of Differentiation (CD) symbols. Extracted CD markers that are known surface markers of immune cells were excluded. Finally, to avoid random mention of the markers, we only selected those that occurred in either the title or abstract of at least two publications (FIG. 1a). Then we ranked them by their occurrence frequency (FIG. 1b). CD133 (also known as PROM1), CD44, CD24 and CD34 are the top 4 most referenced CD markers in lung TIC associated studies.

### B. A Function-Specific Association Study Identifies Robust Correlation Between CD133 and OXTR

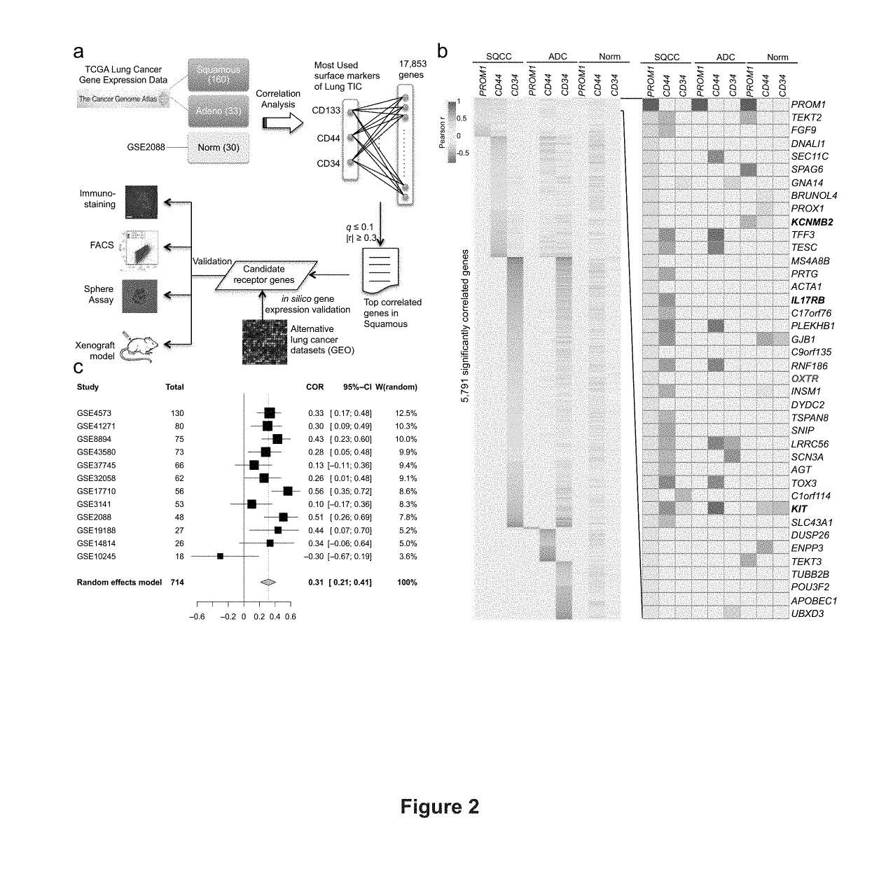

To perform a correlation analysis using these marker genes as seeds, we acquired the gene expression profiles of SQCC and ADC from The Cancer Genome Atlas (TCGA) (TCGA. https://tcga-data.nci.nih.gov/tcga/.(2012)) (FIG. 2a). To control for non-tumor related correlation, we also acquired gene expression datasets of normal lung from Gene Expression Omnibus (GEO). Since the TCGA microarray datasets do not include the gene expression profile of CD24, we chose CD133/PROM1, CD44 and CD34 as the seeds for the association study. We found that significantly correlated genes were exclusively distinctive between the seeds. Similarly, correlated genes with the same seed were different among SQCC, ADC and normal lung tissue (FIG. 2b right). Among the top associated genes with PROM1 (FIG. 2b right) was known lung TIC associated factor KIT (c-kit) (Levina et al, “Elimination of human lung cancer stem cells through targeting of the stem cell factor-c-kit autocrine signaling loop,” Cancer research (2010) 70: 338-346) and transcription factor POU3F2 (Oct-7) (Ishii et al., “Class III/IV POU transcription factors expressed in small cell lung cancer cells are involved in proneural/neuroendocrine differentiation,” Pathology international (2014) 64: 415-422). With a focus on finding new functional TIC specific surface markers, we further searched for all genes coding for receptors that were significantly correlated (q≦0.1, |r|≧0.3) with PROM1 in SQCC. OXTR (Oxytocin receptor) was the 3rd ranked positively correlated gene, after KCNMB2 (Charybdotoxin receptor subunit beta-2) and IL17RB (interleukin-17 receptor B), and followed by other known cancer associated receptors, such as KIT, CLDN3 (Clostridium perfringens enterotoxin receptor 2), and ERBB4 (Receptor tyrosine-protein kinase erbB-4). However, only OXTR was exclusively correlated with PROM1 in SQCC, not CD44 or CD34 and not in ADC or normal lung (FIG. 2b right). Notably, some known lung cancer drug targets, such as EGFR and DDR1, were negatively correlated with PROM1.

To further validate the correlation between CD133 and OXTR in SQCC, we acquired alternative gene expression datasets from GEO. Twelve independent datasets totaling 714 human SQCC samples of published studies were chosen for a meta-analysis of this correlation in SQCC. The meta-correlation coefficient was 0.31 with a p value of 3.10E-09 (FIG. 2C). This correlation of CD133 with OXTR was not seen with vasopressin receptors (AVR), which share ligand-binding activity with OXTR (Kimura et al., “Structure and expression of a human oxytocin receptor,” Nature (1992) 356: 526-529; Sugimoto et al., “Molecular cloning and functional expression of a cDNA encoding the human V1b vasopressin receptor,” The Journal of biological chemistry (1994) 269: 27088-27092; Thibonnier et al., “Molecular cloning, sequencing, and functional expression of a cDNA encoding the human V1a vasopressin receptor,” The Journal of biological chemistry (1994) 269: 3304-3310). This robust and specific correlation, in addition to some evidence that suggests OXTR may promote tumor growth in lung cancer (Pequeux et al., “Oxytocin synthesis and oxytocin receptor expression by cell lines of human small cell carcinoma of the lung stimulate tumor growth through autocrine/paracrine signaling,” Cancer research (2002) 62: 4623-4629; Pequeux et al., “Oxytocin- and vasopressin-induced growth of human small-cell lung cancer is mediated by the mitogen-activated protein kinase pathway,” Endocrine-related cancer (2004) 11: 871-885), led us to further empirically test its validity and determine its association with TIC growth.

C. CD133 is Co-Expressed with OXTR in Human SQCC Tissue, Cell Lines and Primary Tumor Cells

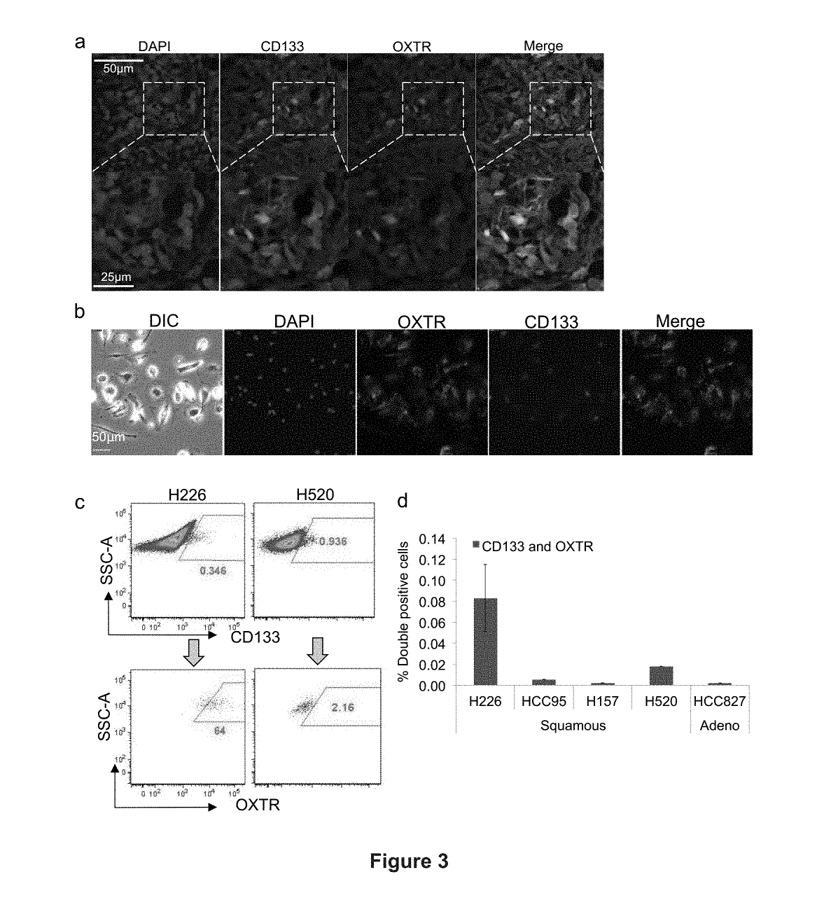

To determine whether the correlation between gene expression of CD133 and OXTR is manifested by the co-expression of the two proteins, we stained CD133 and OXTR in FFPE (formalin fixed paraffin embedded) tumor tissue samples of human SQCC (N=5) using immunofluorescence (IF) staining. Moderate expression of OXTR was detected, which is consistent with its expression in primary lung cancer (Pequeux et al., “Oxytocin receptor pattern of expression in primary lung cancer and in normal human lung,” Lung cancer (2005) 50: 177-188), and low expression of CD133 was detected (FIG. 3a). CD133 was always co-stained with OXTR, but not vice versa. To determine the specificity of this co-staining, tissue samples were also stained for CD44 and immuno-isotypes. CD44 showed higher expression than OXTR, but was not co-stained with OXTR. None of the isotype controls gave IF signals. The expression of CD133 and OXTR was also negative in ADC samples (N=5) and normal lung samples (N=5). Further, in primary cells of SQCC, we detected expression of CD133 and OXTR (FIG. 3b). These results indicated that there was a small fraction of cells in SQCC tumors that co-expressed CD133 and OXTR proteins.

To characterize the expression of these two proteins at a single-cell level, we examined the cell surface protein expression of CD133 and OXTR individually in cell lines of SQCC using fluorescence-activated cell sorting (FACS). In H226, HCC95, H157 and H520 cell lines of SQCC, 0.01% to 1.2% of the cells were CD133 positive, and 0.03% to 6.6% of the cells were OXTR positive. The H226 cell line had the highest level of OXTR, while the HCC95 cell line had the highest level of CD133. In HCC827 and H522 cell lines of ADC, CD133 positive cells were 0.4% and 0.02%, respectively, and OXTR positive cells were 0.9% and 0.03%, respectively. In normal lung (Schiller & Bittner, “Loss of the tumorigenic phenotype with in vitro, but not in vivo, passaging of a novel series of human bronchial epithelial cell lines: possible role of an alpha 5/beta 1-integrin-fibronectin interaction,” Cancer research (1995) 55: 6215-6221), CD133 positive cells were 0.02%, while OXTR positive cells were 1.8%. These results indicate that the expression of CD133 and OXTR varied greatly among cell lines of SQCC, and low expression of each protein was also seen in cell lines of ADC and normal lung.

Next we quantified the co-expression of CD133 and OXTR in each of the cell lines. CD133 and OXTR double positive cells were only found in H226, HCC95, H157, H520 and HCC827 cell lines (FIGS. 3c and 3d). The percentage of the double positive cells ranged from 0.002% to 0.08% (FIG. 3d), which is comparable with the frequency of TIC found in blood tumors (Bonnet & Dick, “Human acute myeloid leukemia is organized as a hierarchy that originates from a primitive hematopoietic cell,” Nature medicine (1997) 3:730-737). H226 and H520 cell lines had more double positive cells than the rest cell lines, and were selected for further study. Together these results suggest that CD133 is co-expressed with OXTR in a small fraction of SQCC cells, but not vice versa.

### D. OXTR is Ubiquitously Expressed in Tumor Sphere Cells of SQCC

To determine the role of co-expression of CD133 with OXTR in lung TICs, we applied a sphere-forming assay to cell lines of SQCC, which is a widely accepted approach to assess the self-renewal potential of neoplastic cells (Reynolds & Weiss, “Generation of neurons and astrocytes from isolated cells of the adult mammalian central nervous system,” Science (1992) 255: 1707-1710) and an effective way to expand TIC populations (Eramo (2008) supra). We isolated OXTR and CD133 double positive (OXTR+/CD133+), OXTR positive and CD133 negative (OXTR+/CD133−), OXTR and CD133 double negative (OXTR−/CD133−) and unsorted single cells using FACS. No OXTR negative and CD133 positive cells could be isolated, which is consistent with the tissue co-staining of CD133 with OXTR.

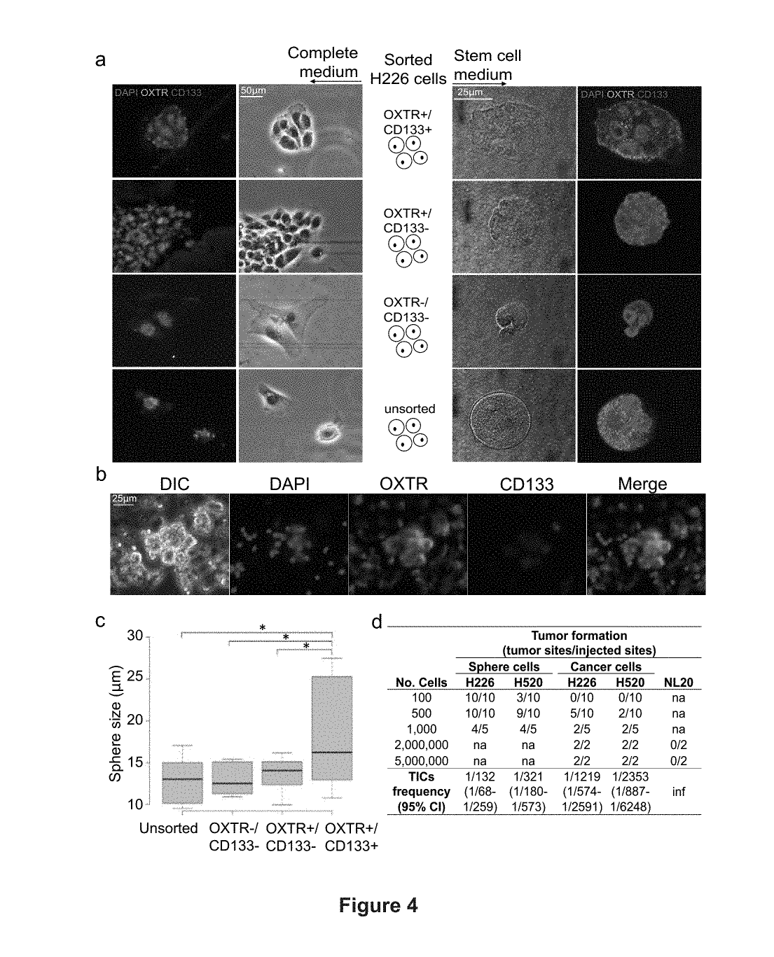

Both sorted and unsorted cells were able to form spheres (FIG. 4a, right) and there was no significant difference in the number of spheres formed. However, the OXTR+/CD133+ cells formed significantly larger spheres (FIG. 4b). To determine the expression of the two proteins in these spheres, we stained for OXTR and CD133 in the spheres formed from sorted and unsorted cells cultured in stem cell medium. OXTR was ubiquitously expressed in all spheres regardless of prior sorting condition, while CD133 only had low expression in spheres formed from OXTR+/CD133+ cells (FIG. 4a). The staining of OXTR was also specific. Neither isotype control nor the fluoro dye-conjugated secondary antibody alone stained the spheres. Further, in tumor spheres derived from primary SQCC tumors, we also detected the ubiquitous expression of OXTR (FIG. 4b). These results indicate that CD133 and OXTR double positive tumor cells tend to form larger tumor spheres in stem cell medium, however, only OXTR expression is ubiquitous in tumor spheres and can be independent of CD133 expression.

To determine the specificity of the expression pattern of OXTR, we proliferated the sorted and unsorted H226 cells in regular cell medium and stained for OXTR and CD133. Tumor cells derived from OXTR+/CD133+ cells expressed OXTR, both at the cell surface and in the cytoplasm, but not all of them expressed CD133 (FIG. 4a, left). Cells derived from OXTR+/CD133− cells, as expected, had OXTR expression but no CD133 expression. Cells derived from OXTR−/CD133− cells, unexpectedly, also had OXTR expression, but in the cytoplasm peri-nuclearly rather than on the cell surface. This could explain why OXTR of these cells were not detected by flow cytometry and were sorted as double negative. For unsorted H226 cells, some of them had OXTR expression on the cell surface and some in the cytoplasm (FIG. 4a, right). These phenomena were also seen with H520 cells. Together these results indicate that cells could lose the expression of CD133 during proliferation; however, OXTR is always expressed on the cell surface or in the cytoplasm of regular tumor cells. This protein expression is also in accordance with the gene expression of OXTR measured by real time quantitative PCR (RT-PCR). We detected the gene expression of OXTR in both regular cancer cells and tumor sphere cells of the H226 and H520 cell lines. Compared to normal lung cells, OXTR was highly overexpressed (4-300 fold) in both regular cancer cells and tumor sphere cells, and no significant difference was detected between the gene expression level in regular cancer cells and tumor sphere cells.

### E. OXTR-Expressed Tumor Sphere Cells of SACC are Tumorigenic

Next we wanted to know whether tumor spheres that express OXTR are tumorigenic or enriched with TICs. Using a limiting dilution analysis in a mouse xenograft model, we assessed the tumorigenicity of the tumor spheres of SQCC cell lines. Since tumor spheres of SQCC cell lines all express OXTR, we used unsorted tumor cells to quickly derive a large amount of tumor spheres for subcutaneous implantation. We found that tumor spheres derived from H226 and H520 cells were nearly 10 fold more tumorigenic than the regular cancer cells (FIG. 4c). As low as 100 tumor sphere cells of either H226 or H520 cell lines were able to initiate tumor growth in vivo, while the same amount of regular tumor cells was not able to (FIG. 4c). Taken together, these results indicate that tumor spheres of SQCC cells are tumorigenic, likely enriched for lung TICs, and ubiquitously express OXTR.

### F. Pharmacological Alteration of OXTR Activity Affects the Growth of Lung TICs

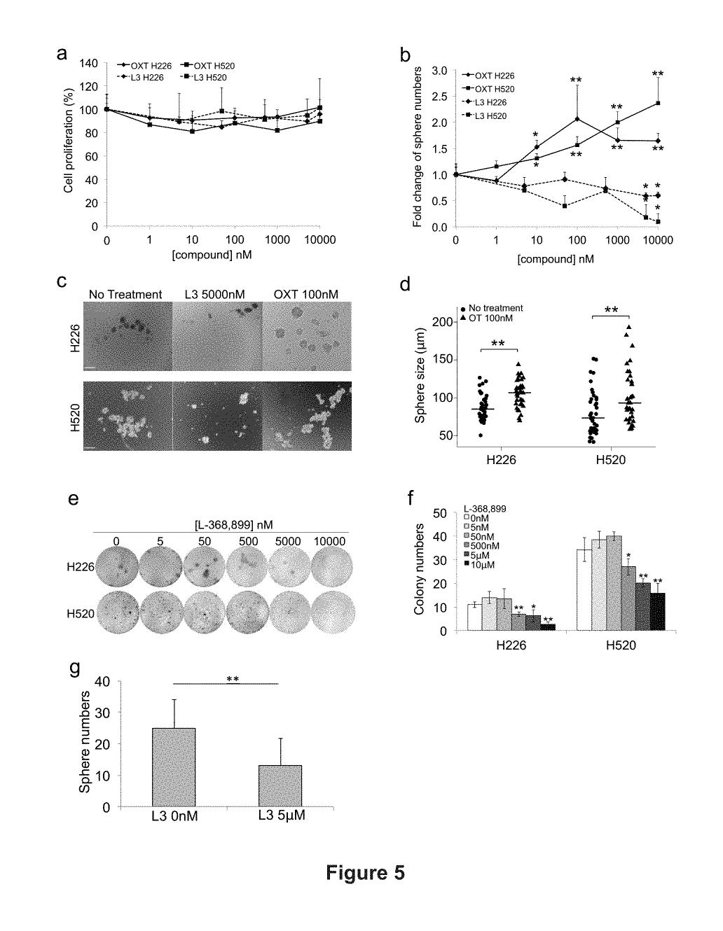

To determine whether the expression of OXTR is associated with the growth of regular cancer cells and TICs, we treated regular cancer cells and tumor spheres of SQCC cell lines with an OXTR specific inhibitor (Manning et al.,“Oxytocin and vasopressin agonists and antagonists as research tools and potential therapeutics,” Journal of neuroendocrinology (2012) 24: 609-628), L-368,899 (L3) to suppress its activity, or its natural ligand oxytocin (OXT) to induce its activity. Although neither L3 nor OXT affected the cell proliferation of regular H226 and H520 cells in a 3-day treatment (FIG. 5a), L3 resulted in dose-dependent decrease of tumor sphere numbers of both H226 and H520 cells after 3 days of treatment, statistically significant at 5 μM and 10 μM (FIG. 5b, 5c). OXT, on the other hand, resulted in dose dependent increase of tumor sphere growth in both cell lines, statistically significant at as low as 10 nM (FIG. 5b, 5c). Tumor spheres treated with OXT were also significantly larger than those treated with solvent control (FIG. 5d). OXTR inhibition by L3 also reduced the colony formation of H226 and H520 cells, statistically significant at 0.5 μM, 5 μM and 10 μM (FIG. 5e, 5f). Further, in tumor spheres derived from primary SQCC cells, L3 significantly reduced the sphere growth at 5 μM (FIG. 5g). These results indicate that the activity of OXTR is specifically associated with the self-renewal and differentiation of lung TICs.

### G. OXTR Knockdown Affects the Tumorigenesis of Lung TICs

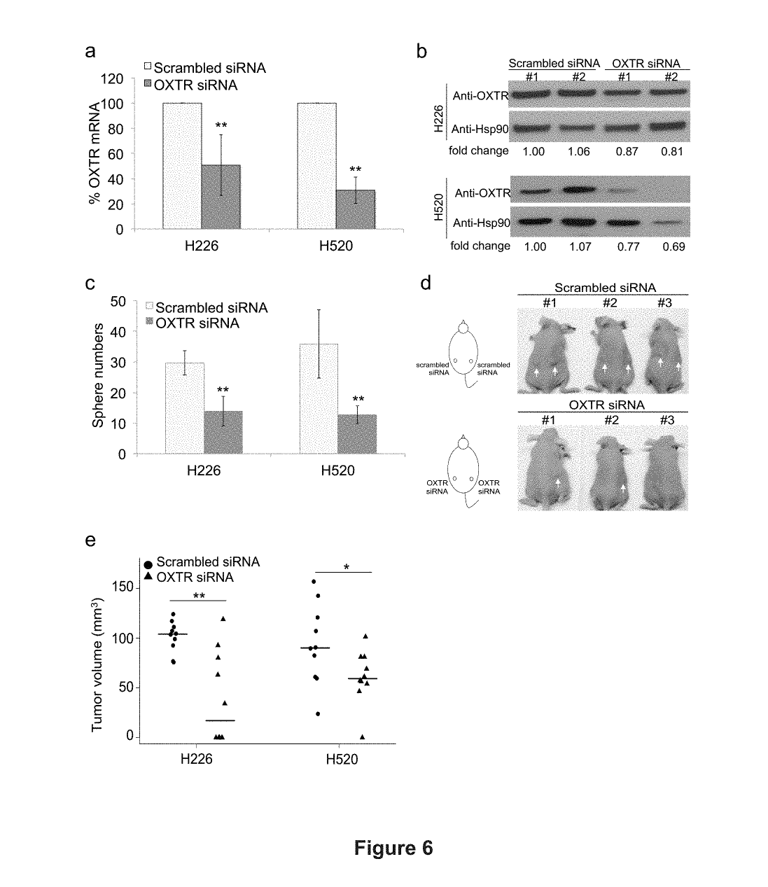

To further determine the role of OXTR in tumorigenesis, we transiently knocked down OXTR in cell lines of SQCC in vitro. Compared to the scrambled siRNA, OXTR siRNA significantly decreased the level of OXTR mRNA in both H226 and H520 cells, with a reduction ranging from 25% to 80% (FIG. 6a). The OXTR protein was reduced only about 13% to 19% (FIG. 6b), however, cells with reduced OXTR derived significantly fewer tumor spheres (FIG. 6c). These results indicate that knockdown of OXTR gene reduces the TIC growth. To determine the knockdown effect on tumor growth in vivo, 4 million H226 cells (or 2 million H520 cells) transfected with either scrambled or OXTR siRNA were subcutaneously implanted in mice (N=20, 10 for each type of siRNA, repeated 3 times). First detectable tumors were formed 3 days after implantation in scrambled control mice and all scrambled control mice had detectable tumors 5 days after implantation (FIGS. 6d and 6e). On the other hand, only half (5 out of 10) of the implantation with OXTR siRNA transfected cells formed tumors 5 days after implantation, and tumor size was significantly smaller (FIG. 6e). Not until 2 weeks after implantation had all cells with OXTR siRNA formed tumors in mice. Thus OXTR knockdown in tumor cells significantly delayed the onset of tumor formation in vivo and reduced the tumor growth (FIGS. 6d and 6e). These results indicate that OXTR activity is associated with in vivo tumorigenesis of SQCC cells.

### H. OXTR Activity in Lung TICs is Mediated by MAPK Pathway

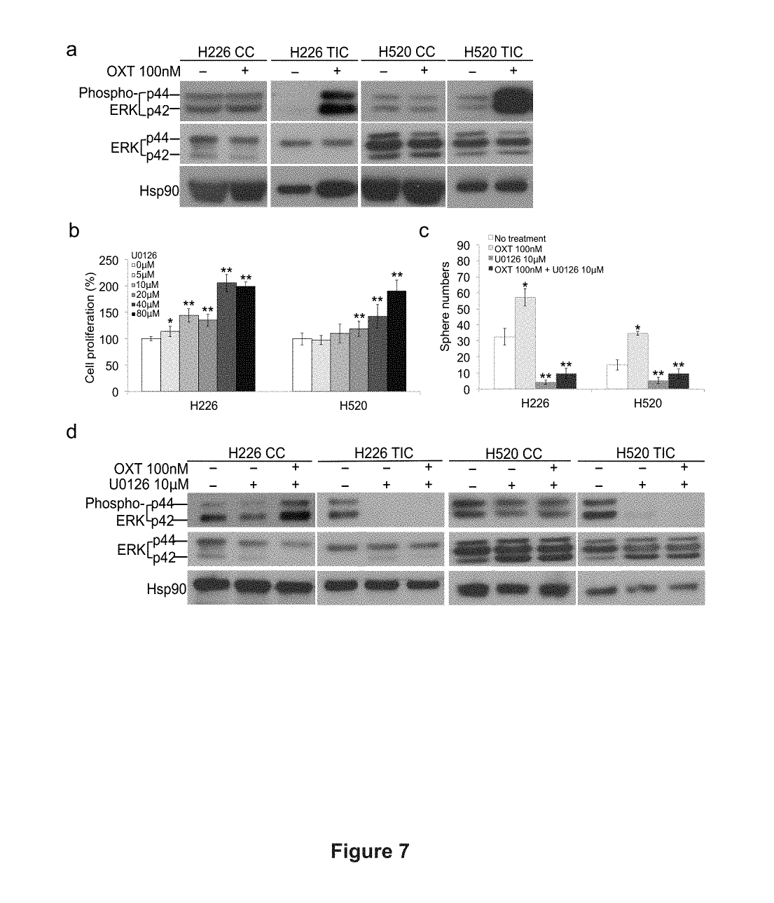

As a G-protein coupled receptor (GPCR), OXTR has been extensively studied for its associated signaling pathways in both normal and tumor tissues (Devost et al., “Oxytocin receptor signalling,” Progress in brain research (2008) 170: 167-176; Carter, “Oxytocin pathways and the evolution of human behavior,” Annual review of psychology (2014) 65: 17-39). It has also been studied in SCLC and was found to promote tumor growth via a mitogen-activated protein kinase (MAPK) pathway (Pequeux (2002) supra; Pequeux (2004) supra). To determine whether the MAPK pathway also mediates OXTR activity in SQCC, we examined the activity of MAP kinases ERK1/2 upon stimulation and inhibition of OXTR in regular cancer cells and tumor spheres of H226 and H520 cell lines. Treated with 100 nM OXT for two days, the phosphorylation of ERK1/2 in TICs was greatly increased compared to solvent control, while it was unchanged in regular H226 and H520 cells (FIG. 7a). This OXT induced TIC-specific activation of ERK1/2 was in accordance with the specific effect of OXT on TIC growth (FIG. 5b). To further validate that the effect of OXTR activity on TIC growth is mediated by the MAPK pathway, we blocked the ERK1/2 activity using an inhibitor of upstream MEK1/2 kinase, U0126. U0126 resulted in dose dependent inhibition of TIC growth, while it increased proliferation in regular cancer cells (FIG. 7b). U0126 also blocked the OXT induced increase of TIC growth in both H226 and H520 cells (FIG. 7c) and inhibited the OXT induced phosphorylation of ERK1/2 (FIG. 7d). This inhibitory effect of U0126 could not be rescued by increasing dose of OXT. These results indicate that MAPK pathway mediates the effect of OXTR activity on the TIC growth of SQCC.

### I. Oxytocin Stimulates Lung TIC Growth Through an Autocrine/Paracrine Signaling

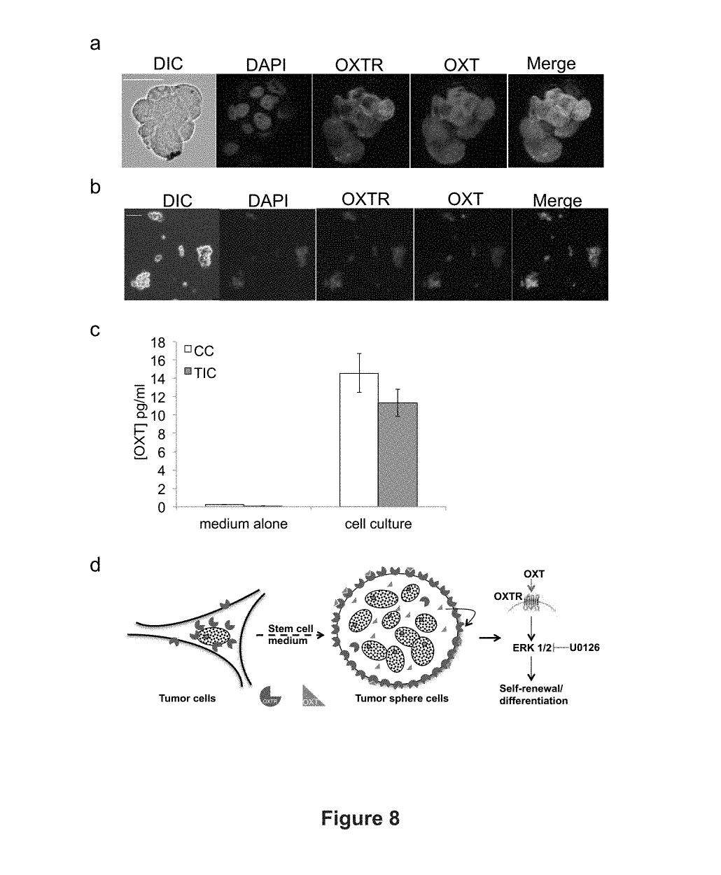

To determine the endogenous resource of OXT for TIC growth, we stained tumor sphere cells for OXTR and OXT. OXT was co-stained with OXTR in tumor spheres of H226 cell line and primary SQCC cells (FIG. 8a). OXT was also secreted into cell culture of both regular cancer cells and TICs (FIG. 8c), both of which express OXTR (FIG. 4a). These results suggest that OXT is produced and secreted by both regular cancer cells and TICs of SQCC, to facilitate an autocrine/paracrine OXT signaling for its growth (FIG. 8d).

### III. Discussion

Here we applied a function-specific guilt-by-association (GBA) approach, where prior knowledge of lung TIC surface markers along with publicly-available gene expression data from The Cancer Genome Atlas (TCGA) were utilized, to identify new functional lung TIC markers. The GBA hypothesis states that genes that are associated or interacting are more likely to share functions (Oliver, “Guilt-by-association goes global,” Nature (2000) 403: 601-603). Although this principle has been exploited by computational biologists as a method for assigning function across a large gene network, most predicted gene associations are never actually tested biologically. Small-scale studies that test the associations of a single genes under more controlled conditions have shown the GBA approach to be more efficient in rejecting spurious findings and enrich for functionally relevant associations (Gillis & Pavlidis, “‘Guilt by association’ is the exception rather than the rule in gene networks,” PLoS computational biology (2012) 8: e1002444).

Putative TIC markers, although nonspecific, have been shown to enrich for cells with TIC properties in different experimental settings. Thus according to the GBA principle, genes associated with putative TIC markers in a specific type of cancer might share similar function and be associated with TIC cells. Our study started from this hypothesis and tested its validity in SQCC. In addition to identifying the genes associated with putative TIC markers, we also discovered the distinctive association patterns between TIC markers and between lung cancer subtypes. Each putative TIC marker has mutually exclusive set of associated genes within a lung cancer subtype (FIG. 1b), which implies that differential gene networks may be associated with different markers. Then for each marker, the set of associated genes was also drastically different between subtypes of lung cancer and normal lung tissue (FIG. 1b). This drastic difference between the association patterns in normal lung and lung cancers implies a reshuffling of cell signaling networks upon malignant transformation.

Our focus on the genes correlated with CD133 in SQCC revealed a new cell surface marker of lung TIC, OXTR, in addition to recovering some of the other known TIC associated markers (FIG. 1b right). This association is both marker specific and tissue specific. The significant correlation of OXTR with CD133 was not seen with other putative markers, and it was only shown in SQCC but not ADC or normal lung. We also examined the correlation of vasopressin receptors (AVR) with CD133. AVR shares ligand-binding activity with OXTR and has three subtypes of AVRs, V1a, V1b and V2. No significant association was found with any of the AVRs.

Further functional validation of the CD133-OXTR association indicated that OXTR expression in our lung TIC model could be independent of CD133 expression (FIG. 4a). The loss of CD133 in TIC cells has also been identified in other tumors, where CD133 negative cells can also develop new tumors (Shmelkov et al., “CD133 expression is not restricted to stem cells, and both CD133+ and CD133− metastatic colon cancer cells initiate tumors,” The Journal of clinical investigation (2008) 118: 2111-2120; Wu & Wu, “CD133 as a marker for cancer stem cells: progresses and concerns,” Stem cells and development (2009) 18: 1127-1134). This also points to the reliability issue of putative markers in representing TICs and suggests OXTR as a more reliable TIC marker.

Although OXTR and OXT, the oxytocinergic system, have been found to be involved in stimulating cell proliferation in a variety of cancers (Cassoni et al., “Oxytocin and oxytocin receptors in cancer cells and proliferation,” Journal of neuroendocrinology (2004) 16: 362-364), including SCLC, we have discovered a new role for this system in the tumorigenesis of SQCC. More importantly, the surface expression of OXTR was acquired and ubiquitous in TIC (FIG. 3a), because even cells sorted for negative expression of OXTR grew into spheres with ubiquitous expression of OXTR. However, the negative FACS signal was not due to the non-expression of OXTR but instead due to the perinuclear trapping of OXTR (FIG. 3a). This cytoplasmic expression could also be the reason of the ineffectiveness of OXTR ligands, OXT and L3, on regulating the growth of regular cancer cells. OXTR expression in TIC was also accompanied by OXT expression, which implied an autocrine/paracrine signaling of OXT for OXTR mediated function as previously shown in SCLC (Pequeux (2002) supra). Moreover, the activity of OXTR was also mediated by the MAPK pathway as it was found in SCLC (Pequeux (2004) supra). Thus, although SQCC is considered a non-neuroendocrine subtype of lung cancer (Friedmann, supra), its TICs may still exploit the same potent autocrine/paracrine signaling pathways to regulate its self-renewal and differentiation (FIG. 5f). Likewise, it was reported that Erythropoietin (a hematopoietic hormone) could promote tumorigenesis of TIC in breast cancer (Zhou et al., “Erythropoietin promotes breast tumorigenesis through tumor-initiating cell self-renewal,” The Journal of clinical investigation (2014) 124: 553-563). This type of hormonal regulation in tumorigenesis might be a common survival tool for TIC.

The lack of any effect of OXTR stimulation or inhibition in regular cancer cell line cells, at least during the 3-day treatment, indicated the ineffectiveness of targeting OXTR in regular SQCC cells, although it has been shown to be effective in SCLC (Pequeux (2002) supra). Thus for SQCC, combining TIC specific targeting with the regular tumor cell inhibition may be required to make the overall treatment effective. Consistently, the inhibition of ERK1/2 activation, which is a signaling pathway of TIC, did not decrease the proliferation of regular cancer cells (FIG. 7b). Further, we found that the gene expression of known targets of lung cancer cells, such as EGFR and DDR1, was reversely correlated with that of CD133. If this reverse correlation is true, then inhibiting these targets could selectively maintain TIC growth, as seen in recurrent lung cancers after chemotherapy (Levina, supra). These findings support a combinatory strategy to targeting both bulk regular cancer cells and TIC populations for reduction of tumor burden and inhibition of tumorigenesis respectively.

Together, our study demonstrated that a controlled GBA analysis that leverages putative TIC markers is an effective approach to identify TIC associated genes, even using heterogeneous cancer genomics data. Our results show that OXTR is a new TIC marker and demonstrate its use as a therapeutic target of SQCC.

### IV. Conclusion

Lung cancer remains the leading cause of cancer death, with frequent disease metastasis and relapse. Tumor initiating cells (TICs) of lung cancer are thought to be a driving force in its development; however currently known surface markers of lung TICs are often non-specific and their role in tumorigenesis are largely unknown. Here we took a function-specific guilt-by-association approach that leverages known putative lung TIC markers for enrichment of more functionally relevant genes. Starting with gene expression data from The Cancer Genome Atlas and the most commonly used lung TIC marker, CD133, we identified a specific and robust association between CD133 and oxytocin receptor (OXTR) in squamous cell lung cancer (SQCC). CD133 was co-expressed with OXTR in tumor tissues, cell lines and primary cells of SQCC, while only OXTR was ubiquitously expressed in TICs of SQCC. Pharmacological inhibition of OXTR reduced TIC growth in cell lines and primary cells of SQCC, while activating it with its ligand oxytocin (OXT) increased TIC growth. Further, OXTR knockdown decreased TIC growth and impaired tumor formation in vivo. Lastly, we found that the mitogen-activated protein kinase pathway mediated the OXTR activity and OXT-stimulated TIC growth through an autocrine/paracrine signaling. Our study provides a new approach to identify TIC specific targets and with this approach we discovered a new role of OXTR in tumorigenesis and its use as a therapeutic target in SQCC.

Our finding provides a new marker, OXTR, for TIC in SQCC, which can be used as a target for drug development and a potential biomarker for the prognosis of lung cancer. OXTR can be used to diagnose NSCLCs as SQCCs, and to identify a TIC population within an SQCC. Therapeutically targeting TIC (e.g., by inhibiting OXTR) wil impair overall NSCLC (e.g., SQCC) growth and development, resulting in better outcomes and less resistance and recurrence. This finding provides a new way to target TIC associated metastasis, recurrence and drug resistance, and improve the outcome of patients with lung squamous carcinoma. For drug development against lung cancer, OXTR can be used as a new target for inhibition of proliferation of cancer stem cell and its associated metastasis, recurrence and drug resistance. To this end, OXTR modulatory agents used therapeutically in the treatment of other conditions (such as preterm labor) can be repurposed. For prognosis of lung cancer, OXTR may be used as a biomarker for predicting the outcome of the patient.

Notwithstanding the appended clauses, the disclosure is also defined by the following clauses:
1. A method of modulating proliferation of a lung squamous cell carcinoma (SQCC) tumor initiating cell (TIC), the method comprising:

contacting the TIC with an amount of an oxytocin receptor (OXTR) modulatory agent effective to modulate the proliferation of the TIC.
2. The method according to Clause 1, wherein the OXTR modulatory agent comprises an OXTR antagonist.
3. The method according to Clauses 1 or 2, wherein the OXTR modulatory agent comprises an oxytocin mimetic.
4. The method according to Clauses 1 or 2, wherein the OXTR modulatory agent comprises a small molecule.
5. The method according to Clause 4, wherein the small molecule is selected from the group consisting of atosiban, retosiban, L-368,889, L-371,257, SSR-126,768, WAY-162,720, and derivatives and combinations thereof.
6. The method according to Clauses 1 or 2, wherein the OXTR modulatory agent comprises an OXTR specific binding member.
7. The method according to Clause 6, wherein the specific binding member comprises an antibody or binding fragment thereof.
8. The method according to Clause 1, wherein the OXTR modulatory agent reduces expression of OXTR.
9. The method according to Clause 8, wherein the OXTR modulatory agent is a RNA.
10. The method according to Clause 9, wherein the RNA is a RNAi agent.
11. The method according to Clause 9, wherein the RNA is a miRNA agent.
12. The method according to any of Clauses 1 to 11, wherein the TIC is contacted with the OXTR modulatory agent in vitro.
13. The method according to any of Clauses 1 to 11, wherein the TIC is contacted with the OXTR modulatory agent in vivo.
14. The method according to any of Clauses 1 to 13, further comprising diagnosing the SQCC prior to the step of contacting.
15. The method according to any of Clauses 1 to 14, further comprising predicting whether proliferation of the TIC may be modulated by an OXTR modulatory agent.
16. The method according to Clause 15, wherein the step of predicting whether proliferation of the TIC may be modulated by an OXTR modulatory agent is based on the expression of OXTR in cells of the SQCC.
17. The method according to any of Clauses 1 to 16, further comprising predicting whether proliferation of the TIC may be modulated by an OXTR modulatory agent, based on OXTR co-expression with a TIC marker in cells of the SQCC.
18. The method according to Clause 17, wherein the TIC marker is CD133.
19. The method according to any of Clauses 1 to 18, wherein the SQCC is a human SQCC.
20. A method of treating a subject for a lung squamous cell carcinoma (SQCC), said method comprising:

administering to the subject an amount of an oxytocin receptor (OXTR) modulatory agent effective to treat the subject for the SQCC.
21. The method according to Clause 20, wherein the OXTR modulatory agent comprises an OXTR antagonist.
22. The method according to Clauses 20 or 21, wherein the OXTR modulatory agent comprises an oxytocin mimetic.
23. The method according to Clauses 20 or 21, wherein the OXTR modulatory agent comprises a small molecule.
24. The method according to Clause 23, wherein the small molecule is selected from the group consisting of atosiban, retosiban, L-368,889, L-371,257, SSR-126,768, WAY-162,720, and derivatives and combinations thereof.
25. The method according to Clauses 20 or 21, wherein the OXTR modulatory agent comprises an OXTR specific binding member.
26. The method according to Clause 25, wherein the specific binding member comprises an antibody or binding fragment thereof.
27. The method according to Clause 20, wherein the OXTR modulatory agent reduces expression of OXTR.
28. The method according to Clause 27, wherein the OXTR modulatory agent is a RNA.
29. The method according to Clause 28, wherein the RNA is a RNAi agent.
30. The method according to Clause 28, wherein the RNA is a miRNA agent.
31. The method according to any of Clauses 20 to 30, further comprising diagnosing the SQCC prior to the step of administering.
32. The method according to any of Clauses 20 to 31, further comprising predicting whether proliferation of the TIC may be modulated by an OXTR modulatory agent, based on the expression of OXTR in cells of the SQCC.
33. The method according to any of Clauses 20 to 32, further comprising predicting whether proliferation of the TIC may be modulated by an OXTR modulatory agent, based on OXTR co-expression with a TIC marker in cells of the SQCC.
34. The method according to Clause 33, wherein the TIC marker is CD133.
35. The method according to any of Clauses 20 to 34, further comprising administering an amount of a cancer chemotherapeutic agent effective to treat the subject for the SQCC.
36. The method according to any of Clauses 20 to 35, wherein the subject is a human.
37. A method of predicting whether a non-small cell lung carcinoma (NSCLC) of a subject may be treated by modulating the oxytocin receptor (OXTR), the method comprising:

evaluating OXTR expression by an NSCLC cell of the subject; and

providing a prediction of whether an OXTR modulatory agent would be effective to treat the subject for the SQCC based on the evaluation.
38. The method according to Clause 37, wherein the step of providing the prediction is based on a comparison of OXTR expression by the NSCLC cell to a reference.
39. The method according to Clause 38, wherein the reference comprises control cells.
40. The method according to Clause 39, wherein the reference comprises standardized beads.
41. The method according to any of Clauses 37 to 40, further comprising evaluating expression of a TIC marker by the NSCLC cell.
42. The method according to Clause 41, wherein the TIC marker is CD133.
43. The method according to Clause 41 or 42, wherein the step of providing the prediction is based on the co-expression of OXTR and the TIC marker by the NSCLC cell.
44. The method according to any of Clauses 37 to 43, wherein the step of evaluating comprises microscopy.
45. The method according to any of Clauses 37 to 44, wherein the step of evaluating comprises flow cytometry.
46. The method according to any of Clauses 37 to 45, wherein the NSCLC is a lung squamous cell carcinoma (SQCC).
47. An in vivo method for visualizing a lung squamous cell carcinoma (SQCC) in a subject, said method comprising:

contacting the SQCC with an oxytocin receptor (OXTR) specific binding member conjugated to a first detectable label; and

detecting a first signal provided by the first detectable label.
48. The method according to Clause 47, wherein the OXTR binding member is selected from oxytocin, an oxytocin mimetic, an OXTR-specific antibody or a fragment thereof.
49. The method according to Clause 47 or 48, wherein the detectable label is selected from a fluorescent dye, a phosphorescent dye, a colorimetric dye, and a radioactive agent.
50. The method according to any of Clauses 47 to 49, further comprising contacting the SQCC with a TIC specific binding member conjugated to a second detectable label.
51. The method according to Clause 50, wherein the TIC specific binding member is a CD133 specific binding member.
52. The method according to Clause 50 or 51, further comprising detecting a second signal provided by the second detectable label.
53. The method according to any of Clauses 47 to 52, wherein the subject is a human.
54. A method of screening an oxytocin receptor (OXTR) modulatory agent effective to treat a subject for a lung squamous cell carcinoma (SQCC), said method comprising:

contacting SQCC cells with a potential OXTR modulatory agent; and

evaluating proliferation of the SQCC cells.
55. The method according to Clause 54, wherein the SQCC cells comprise TIC.
56. The method according to Clause 55, wherein the TIC co-expresses OXTR and CD133.
57. The method according to any of Clauses 54 to 56, wherein the step of evaluating comprises contacting the SQCC cells with a proliferation assay dye prior to culturing the SQCC cells in vitro.
58. The method according to any of Clauses 54 to 57, wherein the step of evaluating comprises performing a sphere assay.
59. The method according to any of Clauses 54 to 58, wherein the step of contacting comprises administering the potential OXTR modulatory agent to an animal model having a lung SQCC.
60. The method according to Clause 59, wherein the step of evaluating comprises comparing the growth of the lung SQCC in the animal model treated with the potential OXTR modulatory agent to a control.
61. The method according to Clause 60, wherein the animal model is a murine model.
62. The method according to any of Clauses 54 to 61, wherein the SQCC cells are human SQCC cells.
63. A pharmaceutical composition, the composition comprising:

an oxytocin receptor (OXTR) modulatory agent; and

an additional anti-cancer active agent.
64. The composition according to Clause 63, wherein the OXTR modulatory agent comprises an OXTR antagonist.
65. The composition according to Clauses 63 or 64, wherein the OXTR modulatory agent comprises an oxytocin mimetic.
66. The composition according to Clauses 63 or 64, wherein the OXTR modulatory agent comprises a small molecule.
67. The composition according to Clause 66, wherein the small molecule is selected from the group consisting of atosiban, retosiban, L-368,889, L-371,257, SSR-126,768, WAY-162,720, and derivatives and combinations thereof.
68. The composition according to any of Clauses 63 to 67, wherein the OXTR modulatory agent comprises an OXTR specific binding member.
69. The composition according to Clause 68, wherein the specific binding member comprises an antibody or binding fragment thereof.
70. The composition according to any of Clauses 63 to 69, wherein the OXTR modulatory agent reduces expression of OXTR.
71. The composition according to Clause 70, wherein the OXTR modulatory agent is a RNA.
72. The composition according to Clause 71, wherein the RNA is a RNAi agent.
73. The composition according to Clause 71, wherein the RNA is a miRNA.
74. The composition according to any of Clauses 63 to 73, wherein the additional anti-cancer active agent comprises a non-proteinaceous compound that reduces proliferation of cancer cells.
75. The composition according to any of Clauses 63 to 74, wherein the additional anti-cancer active agent comprises a chemotherapeutic agent.
76. The composition according to Clause 75, wherein the chemotherapeutic agent comprises a cytotoxic agent.
77. The composition according to Clause 75, wherein the chemotherapeutic agent comprises a DNA alkylating agent.
78. The composition according to Clause 75, wherein the chemotherapeutic agent comprises an antimetabolite.
79. The composition according to Clause 75, wherein the chemotherapeutic agent is selected from Cisplatin, Carboplatin, Paclitaxel, Albumin-bound paclitaxel, Docetaxel, Gemcitabine, Vinorelbine, Irinotecan, Etoposide, Vinblastine and Pemetrexed.
80. The composition according to any of Clauses 63 to 79, further comprising a pharmaceutically acceptable carrier.
81. A kit comprising:

an oxytocin receptor (OXTR) specific binding member conjugated to a first detectable label; and

a TIC specific binding member conjugated to a second detectable label.
82. The kit according to Clause 81, wherein the OXTR specific binding member is oxytocin, an oxytocin mimetic, an OXTR antibody or a fragment thereof.
83. The kit according to Clauses 81 or 82, wherein the TIC specific binding is a CD133 specific binding member.
84. The kit according to any of Clauses 81 to 83, wherein one or both of the first and second detectable labels is selected from a fluorescent dye, a phosphorescent dye, a colorimetric dye, and a radioactive agent.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of modulating a lung squamous cell carcinoma (SQCC) tumor initiating cell (TIC), the method comprising:
contacting the TIC with an amount of an oxytocin receptor (OXTR) modulatory agent effective to modulate the TIC.

2. The method according to claim 1, wherein the OXTR modulatory agent comprises an OXTR antagonist.

3. The method according to claim 2, wherein the OXTR modulatory agent comprises an oxytocin mimetic, a small molecule or an OXTR specific binding member.

4. The method according to claim 1, wherein the OXTR modulatory agent reduces expression of OXTR.

5. The method according to any of claims 1 to 4, wherein the TIC is contacted with the OXTR modulatory agent in vitro.

6. The method according to any of claims 1 to 4, wherein the TIC is contacted with the OXTR modulatory agent in vivo.

7. The method according to claim 6, wherein the TIC is present in a subject suffering from SQCC.

8. The method according to claim 7, wherein the method further comprises administering a tumor burden reduction therapy to the subject.

9. The method according to claim 8, wherein the tumor burden reduction therapy comprises administering an anti-cancer active agent to the subject.

10. The method according to any of claims 7 to 9, further comprising diagnosing the present of SQCC in the subject.

11. The method according to any of claims 7 to 10, further comprising predicting whether proliferation of the TIC may be modulated by an OXTR modulatory agent.

12. The method according to any of claims 7 to 11, wherein the method is a method of treating the subject for SQCC.

13. The method according to any of claims 7 to 12, wherein the subject is human.

14. A pharmaceutical composition for the treatment of a lung squamous cell carcinoma (SQCC) in a subject, the composition comprising:
an oxytocin receptor (OXTR) modulatory agent; and
an additional anti-cancer active agent.

15. The pharmaceutical composition according to claim 14, wherein the additional anti-cancer active agent is a chemotherapeutic agent.
